(19) United States
(12) Patent Application Publication
(10) Pub. No.: US 2006/0057578 A1
(43) Pub. Date: Mar. 16, 2006

(54) Determination of an analyte in a liquid medium

(75) Inventors: Itamar Willner, Mevasseret Zion (IL); Yossi Weizmann, Ramat HaGolan (IL); Pernando Patolsky, Cambridge, MA (US)

Correspondence Address: BROWDY AND NEIMARK, P.L.L.C.;624 NINTH STREET, NW, SUITE 300, WASHINGTON, DC, 20001-5303, US

(73) Assignee: Yissum Research Development Company of the Hebrew University of Jerusalem, Jerusalem (IL)

(21) Appl. No.: 10/513,455

(22) PCT Filed: May 6, 2003
(86) PCT No.: PCT/IL03/00369

§ 371 Date: Jul. 20, 2005

Related U.S. Application Data
(60) Provisional application No. 60/378,410, filed on May 8, 2002.
(60) Provisional application No. 60/436,005, filed on Dec. 26, 2002.

(51) Int. Cl. C12Q 1/68; G01N 33/551

(52) U.S. Cl. 435/6; 436/524

(57) ABSTRACT

The present invention concerns a magneto-controlled method and system for the determination of an analyte in a liquid medium. The method and system of the invention are based on the use of functionalized magnetic particles, e.g. magnetic particles that carry a recognition agent, such that in the presence of the analyte and under appropriate conditions, a chemical reaction occurs yielding a reaction signal. The reaction signal may be an electric signal, a colorimetric signal, light emission or the formation of a precipitate. In accordance with the invention the reaction is significantly enhanced by inducing rapid vibrations or rotations of the magnetic particles on the barrier surface.

### FIELD OF THE INVENTION

This invention relates to a method for detecting an analyte in an assayed sample. More specifically, the present invention concerns a magneto-controlled method for determination of an analyte in a liquid medium.

### LIST OF REFERENCES

The following references are considered to be pertinent for the purpose of understanding the background of the present invention:
1. Hirsch, R.; Katz, E.; Williner, I.; J. Am. Chem. Soc. 2000, 122, 12053-12054.
2. Katz, E.; Sheeney-Haj-Ichia, L.; Wiliner, I., Chem. Eur. J. 2002, 8, 4138-4148.
3. Katz, E.; Sheeney-Haj-Ichia, L.; Buckmann, A. F.; WilIner, I.; Angew. Chem. Int. Ed. 2002, 41, 1343-1346.
4. Sheeney-Haj-Ichia, L.; Katz, E.; Wasserman, J.; Willner, I.; Chem. Commun. 2002, 158-159.
5. Katz, E.; Willner, I.; Electrochem. Commun. 2002, 4,201-204.
6. Dickson, D. P. E.; Walton, S. A.; Mann, S.; Wong, K.; NanoStruct. Mater. 1997, 9, 595-598.
7. De Cuyper, M.; Joniau, M.; Biotechnol. Appl. Biochem. 1992, 16, 201-210.
8. Carpenter, E. E.; J. Magnetism Magnetic Mater. 2001, 225, 17-20.
9. Matsunaga, T.; Takeyama, H.; Supramolec. Sci. 1998, 5, 391-394.
10. Liao, M.-H.; Chen, D.-H.; Biotechnol. Lett. 2001, 23, 1723-1727.
11. Mornet, S.; Vekris, A.; Bonnet, J.; Duguet, E.; Grasset, F.; Choy, J.-H.; Portier, J.; Mater. Lett. 2000, 42, 183-188.
12. Sonti, S. V.; Bose, A.; J. Colloid Interface Sci. 1995, 170, 575-585.
13. Shen, L.; Laibinis, P. E.; Hatton, T. A.; Langmuir 1999, 15, 447453.
14. Katz, E.; Lotzbeyer, T.; Schlereth, D. D.; Schuhmann, W.; Schmidt, H.-L.; J. Electroanal. Chem. 1994, 373,189-200.
15. Bard, A. J.; Faulkner, L. R.; Electrochemical Methods: Fundamentals and Applications, Wiley, New York, 1980.
16. Moiroux, J.; Elving, P. J.; J. Am. Chem. Soc. 1980, 102, 6533-6538.
17. Gorton, L.; J. Chem. Soc., Faraday Trans. 1, 1986, 82, 1245-1258.

The above publications will be referenced bellow by indicating their number from the above list.

### BACKGROUND OF THE INVENTION

Recent efforts are directed to the magnetic-field switching of electrocatalytic and bioelectrocatalytic processes.^{1,2} Several applications of magneto-controlled electron transfer reactions, such as selective dual biosensing,³ stimulated electrogenerated chemiluminescence⁴ and selective patterning,⁵ were suggested. Magnetic particles functionalized with chemical or biological components are extensively used as a “collection tool” for the concentration and the localization of chemical or biochemical components.⁶⁻⁹ Different applications of magnetically-confined chemical components were reported, including transport and concentration of enzymes,¹⁰ DNA¹¹ or cells.¹²

### SUMMARY OF THE INVENTION

The present invention provides a method and system for the determination of an analyte in an assayed, liquid sample. The method and system of the invention are based on the use of functionalized magnetic particles, e.g. magnetic particles that carry a recognition agent, such that in the presence of the analyte and under appropriate assay conditions, a reaction occurs yielding a reaction signal.

The term “reaction” is used to denote one or more reactions or interactions carried out at once or in sequence, to yield the reaction signal. The “reaction signal” is any detectable parameter that is yielded by the reaction. Accordingly, the term “assay conditions” encompasses all the conditions, substances or actions necessary or useful for the appropriate reaction to take place, including sequences of varying conditions or actions.

The particles are drawn to a barrier surface in the reaction cell, through a magnet placed in proximity to the barrier surface. The reaction is detected by a sensing member, which forms the barrier surface or is part of the barrier surface, or is located in proximity to the barrier surface or elsewhere.

The sensing member may be an electrode of an electrochemical cell and the reaction signal in such example is an electric response that results from a reaction occurring as a result of the presence of an analyte in the assayed sample. The term “electric response” refers to any measurable change in the electrical parameters recorded by or electrical properties of the electrode. An electric response may be flow of current, charge or potential change, that results from a reaction occurring at the surface of the electrode; a change in the amperometric response of the electrode that can be measured, for example, by means of a cyclical voltamogram; etc. As will no doubt be appreciated, the invention is not limited by the manner in which the electric response is measured and any manner of measurement that may be used therefor could be applied for measurement of the electric response in the method and system of the invention.

In addition to an electric response, other examples for the reaction signal are the emission of light, a colorimetric response or the formation of a precipitate on the sensing member. Such responses may be measured by appropriate optical sensing means. The formation of a precipitate on the sensing member may also be determined through measuring of a change in the electric response of the sensing member, being in such case an electrode, for example using Faradaic impedance spectroscopy.

In accordance with the invention the reaction may be significantly enhanced by inducing rapid movements, i.e. rapid vibrations or rotations of the magnetic particles on the barrier surface. This may be achieved, for example, by a rotating motor associated with the magnet and that causes the magnet to rotate, and hence induces rotation of the magnetic particles.

The electrocatalytic and bioelectrocatalytic transformations at the particles' interface are controlled, among others, by the rate of transport of the analyte or of other substances that participate in the assay, towards the reaction site. Without wishing to be bound by theory, it is believed that the rotation or vibration of the magnetic particles yields a hydrodynamic mass-transport of the analyte and/or assay substances towards the reaction site to facilitate the reaction between the analyte and/or assay substances and the functionalized magnetic particles or any moiety attached thereto. Rotating or vibrating the magnetic particles through a rotating or vibrating magnetic field, is a preferred embodiment of the invention.

The invention permits the qualitative detection of the presence of an analyte in an assayed sample by monitoring the occurrence of a reaction signal. By measuring the extent of the signal, the concentration of the analyte in the assay sample may also be quantitatively determined. In the following, the term “determination” or “determining” or “detection” will be used to refer collectively to both qualitative and quantitative assay of the analyte in the assayed sample.

The term “magnet” will be used to denote both a passive magnet made of a magnetized metal alloy and an electromagnet.

According to one aspect of the invention, there is provided a method for determining an analyte in an assayed sample, comprising:
(i) providing magnetic particles carrying a recognition agent that binds to or reacts with the analyte, such that, under assay conditions, said binding or reaction yields a reaction signal;
(ii) contacting said magnetic particles with the assayed sample, drawing the magnetic particles to a barrier surface through a magnet proximal to the barrier surface, providing the assay conditions and inducing the magnetic particles to rapidly rotate or vibrate, giving rise to a reaction signal; and
(iii) reading said reaction signal.

The magnetic particles used in the method of the invention are typically made of Fe₃O₄, Fe, Co, Ni, their alloys, as well as other ferromagnetic materials.

According to another aspect, the present invention provides a system for determining an analyte in an assayed sample, the system comprising:
(a) a cell with a barrier surface;
(b) a sub-system for causing the magnetic particles to rotate or vibrate;
(c) magnetic particles having immobilized thereon a recognition agent such that in the presence of the analyte, a reaction occurs yielding a reaction signal, said signal being enhanced during the rotation or vibration of said magnet;
(d) sensing member for sensing said reaction signal; and
(e) reader for reading said reaction signal.

Said sub-system, according to one embodiment of the invention, comprises a motor associated with the magnet that causes the magnet to rapidly rotate or vibrate.

The magnetic particles used in the system of the invention are typically made of Fe₃O₄, Fe, Co, Ni, their alloys, as well as other ferromagnetic materials.

According to one embodiment of the invention the system is an electrochemical system and the reaction that yields said reaction signal is a redox reaction.

The present invention is not limited by the nature of the recognition agent and the analyte, the nature of the reaction that yields the reaction signal, the assay conditions or by the reaction signal. There are many types of reactions that permit detection of an analyte in a medium through immobilized recognition agents, such as those disclosed in WO 97/45720 and WO 00/32813 the contents of which are incorporated herein by reference.

In accordance with one embodiment of the invention, the assayed sample is first reacted to cause binding of the analyte, if present in the sample, with a recognition agent which may be a fluorescent or another calorimetric marker, a radio label, an enzyme that can catalyze a detectable reaction or a reagent that can undergo a redox reaction.

Accordingly, the recognition agent and the analyte can react with one another in a manner to yield a reaction product. The reaction is typically, but not exclusively, a redox reaction. The assay conditions, in accordance with this embodiment, comprise temperature conditions and reagents that permit the reaction to occur. The reagents that permit the reaction between the recognition agent and the analyte typically include a catalyst, for example, an enzyme that can catalyze this reaction. Specific examples of analytes that can be detected in accordance with this embodiment include sugar molecules such as glucose, fructose, mannose, etc.; hydroxy or carboxy compounds, e.g. lactate, ethanol, methanol, formic acid, etc.; or amino acids. The recognition agents in such cases are quinones, e.g. naphthoquinones, pyrroloquinoline quinone (PQQ), etc. An enzyme that can induce a reaction, in this case a redox reaction, includes glucose oxidase, lactate dehydrogenase, fructose dehydrogenase, alcohol dehydrogenase cholin oxidase and the like.

In accordance with another embodiment, the recognition agent comprises a catalyst that can induce a reaction in which the analyte is converted into a product. In accordance with this specific embodiment, the reaction may be a redox reaction and the reaction may be monitored through measuring the electric response of an electrode. Where the catalyst is an enzyme, the identity of the enzyme determines specificity of the reaction.

Alternatively, the analyte may be a catalyst that can induce a reaction in which the recognition agent is converted into a product. Accordingly, the reaction signal would be such that is present only if the recognition agent was converted by the catalyst.

In accordance with yet another embodiment of the invention, the analyte and the recognition agent form a recognition pair. Examples of recognition pairs may be: antigen-antibody, ligand-receptor, oligonucleotide-oligonucleotide with a complementary sequence, oligonucleotide-binding protein, and sugar-lectin. The analyte is then one of the pair and the detection moiety the other. The detection may be based on the use of a reagent that binds to the formed couple, such as an agent that binds specifically to a double-stranded oligonucleotide and not to a single-stranded oligonucleotide, or an enzyme that uses only double-stranded oligonucleotides and not single-stranded oligonucleotides as substrates.

In the alternative, detection may be based on a reagent that binds specifically to the analyte. In the latter case, the binding between the analyte and the reagent is permitted first to occur and thereafter, excess reagents are removed and the reaction is allowed to proceed. The reagent may be contacted with the analyte before, during or after the recognition agent is introduced. Examples of such reagents are an antibody or a nucleotide chain, capable of specific binding to the analyte when it is bound to the recognition agent. The reagent may carry a detectable label, which may be a fluorescent, colorimetric or redox label, or may be an agent that can by itself undergo a reaction or catalyze a reaction such as an enzyme, an agent that can undergo a redox reaction, etc.

In the method of the invention, during the analysis, at least one of the components of the chemical system, for example the analyte, the recognition moiety or the catalyst, should be dissolved in the analyzed liquid medium, whereas the remaining component should be linked to the magnetic particles.

In accordance with yet another embodiment of the invention, the assay comprises a first reagent capable of modifying the analyte, or a complex comprising the analyte, such that the reaction product is detectable by a second reagent or more, ultimately yielding a reaction signal that is dependant on the presence or concentration of the analyte in the sample. One example of such assay is use of an enzyme to modify the recognition agent in the presence of the analyte by binding a biotin-containing moiety to the recognition agent. The biotin moiety bound to the recognition agent then serves as a specific binding site to a second reagent comprising for example avidin-horseradish peroxidase (HRP) that acts as a biocatalytic label. It is appreciated that this assay can lead also to amplification of the signal, by repeatedly labeling more than one molecule of the recognition moiety, such as using the polymerase chain reaction to label a recognition agent being single-stranded DNA in the presence of a DNA analyte.

In accordance with another embodiment of the invention a method is provided for the detection of cancer cells comprising:
(i) providing magnetic particles carrying a DNA recognition agent that serves as a primer for telomerase, such that, under assay conditions, the telomerase reaction enables a reaction that yields a reaction signal;
(ii) providing an assay sample comprising cellular extract from one or more cells suspected of being cancerous;
(iii) contacting said magnetic particles with the assayed sample, drawing the magnetic particles to a barrier surface through a magnet proximal to the barrier surface, providing the assay conditions and inducing the magnetic particles to rapidly rotate or vibrate, giving rise to a reaction signal;
(iv) reading said reaction signal; and
(v) comparing said reading with a reading obtained from a control assay sample not containing cancerous cells, a higher reading in the assay sample than in the control assay sample indicating that said suspected cells are cancerous.

It is appreciated that according to this embodiment of the invention, cancer can be detected in tissue taken from a patient, in order to diagnose the patient's condition. Alternatively such tissue samples can be taken during treatment of a known cancer patient in order to evaluate the success or progress of the treatment. The term ‘cancer’ or ‘cancerous’ are used to denote any cancerous or malignant condition of a cell or a patient, whether human or not.

In the method of the invention, the presence of the analyte in the medium results in the formation of a signal, e.g. electrical signal, color signal, light emission or formation of a precipitate, thereby indicating the presence of the analyte. The sensing member is such that can sense the reaction signal. When the signal is emission of light the detector is a light detector.

When the signal is electrical, it results from the transfer of electrons between an electrode and an electron transfer chain, where the analyte is a member of that electron transfer chain.

Electrodes suitable for use in the method of the invention are made of or coated with conducting or semi-conducting materials, for example gold, platinum, palladium, silver, carbon, copper, indium tin oxide (ITO), etc.

It would be appreciated that the methods and systems of the invention are applicable also to the simultaneous or sequential detection of more than one analyte. In such case, the magnetic particles would carry more than one recognition agent (either on the same magnetic particle or on different magnetic particles). In order for simultaneous detection to take place, the assay conditions should be such that would allow the simultaneous formation of reaction signals that are distinguishable for each analyte. Accordingly, the presence of one analyte would lead to a reaction signal of one type (e.g. light emission) while the presence of another analyte would lead to a reaction signal of another type (e.g. formation of a precipitate on a sensing member, or emission of light in a different spectrum). Alternatively, the detection of the more than one analytes may be achieved in sequence, such that after one assay is performed, the magnetic particles are collected, washed and provided with different assay conditions for the detection of another analyte. In such case, the reaction signal may be the same, provided that in each assay the reaction signal would be obtained solely in connection with the presence of a single analyte.

### BRIEF DESCRIPTION OF THE DRAWINGS

In order to understand the invention and to see how it may be carried out in practice, several preferred embodiments will now be described, by way of non-limiting examples and with reference to the accompanying drawings, in which:

FIG. 1 illustrates the functionalization of the magnetic particles with pyrroloquinoline quinone (PQQ) (1) or with N-(ferrocenylmethyl)aminohexanoic acid (2).

FIG. 2A shows cyclic voltammograms of an Au-electrode with the magnetically attracted PQQ-functionalized magnetic particles (10 mg) in the presence of 50 mM NADH upon rotation of the magnet: (a) 0 rpm, (b) 10 rpm, (c) 100 rpm, (d) 1000 rpm. Potential scan rate, 5 mV·s⁻¹.

FIG. 2B shows calibration plots for the amperometric detection of NADH (E=0.1 V) upon rotation of the magnet: (a) 0 rpm, (b) 100 rpm, (c) 1000 rpm. The data were recorded in 0.1 M Tris-buffer, pH 7.0 with 20 mM CaCl₂.

FIG. 3A shows cyclic voltammograms of a Au-electrode with the magnetically attracted (2)-functionalized magnetic particles (6 mg) in the presence of glucose oxidase, 1×10⁻⁵ M, and glucose, 50 mM upon rotation of the magnet: (a) 0 rpm, (b) 10 rpm, (c) 100 rpm, (d) 400 rpm. Potential scan rate, 5 mV·s⁻¹.

FIG. 3B shows calibration plots for the amperometric detection of glucose (E=0.5 V) upon rotation of the magnet: (a) 0 rpm, (b) 100 rpm, (c) 400 rpm. The data were recorded in 0.1 M phosphate buffer, pH 7.0.

FIG. 4A illustrates an embodiment where the functionalization of magnetic particles is made with a DNA primer.

FIG. 4B illustrates a system implementing the embodiment illustrated in FIG. 4A, where the reaction signal is read by means of light emission.

FIG. 5 illustrates a system implementing an embodiment for the functionalization of the magnetic particles, similar to that illustrated in FIG. 4A but where the reaction signal is read by means of Faradaic impedance.

FIG. 6A illustrates the functionalization of magnetic particles with an antigen.

FIG. 6B illustrates the functionalization of magnetic particles with a naphthoquinone (4).

FIG. 6C illustrates an immunosensing system implementing both the embodiments illustrated in FIGS. 6A and 6B.

FIG. 7A shows a plot of the intensity of the light emission vs. time, obtained in the system illustrated in FIG. 6C, without rotation of the magnetic particles (curve a) and with rotation (100 rpm, curve b).

FIG. 7B shows two calibration plots for the light signal intensity vs. the DNP-antibody concentration in the system illustrated in FIG. 6C: (a)—without rotation; and (b)˜with rotation (100 rpm).

FIG. 8A schematically illustrates the binding of a DNA analyte by use of DNA-functionalized magnetic particles, biotin labeled DNA and an avidin-HRP conjugate.

FIG. 8B shows a detection system for a DNA analyte, implementing the DNA-functionalized magnetic particles illustrated in FIG. 8A together with quinone-modified magnetic particles.

FIG. 9A shows a graph of chemiluminescence intensities upon the analysis of a DNA analyte (13), 1.4×10⁻⁸ M, according to FIGS. 8A and 8B, at different rotation speeds: (a) 0 r.p.m.; (b) 60 r.p.m.; (c) 400 r.p.m.; (d) 2000 r.p.m.; (e) Analysis of mutant (13a), 1×10⁻⁷ M, at 2000 r.p.m. Inset: a graph showing the relation between the light intensity and ω² (ω=rotation speed). The chemiluminescence signals are produced by applying a potential step on the electrode from 0 to −0.5 V and back (vs. SCE).

FIG. 9B shows a graph of light intensities as a function of the concentration of a DNA analyte (13) according to FIGS. 8A and 8B, at variable rotation speeds: (a) 0 r.p.m.; (b) 60 r.p.m.; (c) 2000 r.p.m. Inset: Enlargement of the results in the lower concentration range. The chemiluminescence signals are produced by applying a potential step on the electrode from 0 to −0.5 V and back (vs. SCE).

FIG. 10 shows amplified detection of viral DNA by multi-labeled rotating magnetic particles: (A) Labeling of the nucleic acid replica on the particles with biotin units using thermal cycles. (B) Generation of amplified chemiluminescence upon rotation of the functionalized magnetic particles on electrode surfaces.

FIG. 11 schematically shows the binding of a DNA primer as a recognition agent to magnetic particles, using the heterobifunctional cross-linker 3-maleimidopropionic acid N-hydroxysuccinimide ester.

FIG. 12 shows a graph of chemiluminescence intensities upon the analysis of M13φ DNA, 8×10⁻⁹M, at different rotation speeds, (a) 0 r.p.m.; (b) 60 r.p.m.; (c) 400 r.p.m.; (d) 2000 r.p.m., and curve (e) chemiluminescence signal upon applying the protocol in the absence of M13φ DNA at 2000 r.p.m. Inset: Chemiluminescence intensities as a function of ω^{1/2} (ω=rotation speed). Chemiluminescence was generated by the application of a potential step from E=0.0V to E=−0.5V and back vs SCE. Arrows in figure indicate the times for switching the potential to −0.5V and to 0.0V, respectively. Data recorded in 0.01M phosphate buffer pH=7.4 that includes luminol, 1×10⁻⁶M, under air.

FIG. 13 shows a calibration curve corresponding to the chemiluminescence intensities upon analyzing different concentrations of M13 φ DNA at: (a) 2000 r.p.m.; (b) 400 r.p.m.; (c) 60 r.p.m.; (d) 20 r.p.m.; (e) 0 r.p.m.

FIG. 14 shows the amplified detection of a single-base-mismatch in DNA using magnetic particles

FIG. 15 depicts a graph of chemiluminescence intensities upon the analysis of a DNA mutant sequence, (18), 1×10⁻⁹M at: (a) 2000 r.p.m.; (b) 400 r.p.m.; (c) 60 r.p.m. The chemiluminescence intensity upon the analysis of the normal sequence, (19), 1.4×10⁻⁶M, at 2000 r.p.m. is shown in curve (d). The conditions for the recording of the chemiluminescence are detailed in FIG. 12.

FIG. 16 shows calibration curves corresponding to the analysis of different concentrations of the mutant (18) at different rotation speeds: (a) 2000 r.p.m.; (b) 400 r.p.m.; (c) 60 r.p.m.; (d) 0 r.p.m. Inset: Enlargement of calibration curves showing the chemiluminescence intensities at low concentrations of (18).

FIG. 17 schematically shows the amplified rapid detection of telomerase activity by multi-labeled rotating magnetic particles. (A) Multi-labeling of magnetic particles with biotin units as a result of the telomerase enzyme activity. (B) Generation of amplified chemiluminescence upon rotation of the biotin-multifunctionalized magnetic particles on electrode surfaces.

FIG. 18A shows chemiluminescence intensities upon the analysis of a 293-kidney cancer cell extract containing 100,000 cells, at different rotation speeds: 0 r.p.m.; 20 r.p.m.; 60 r.p.m.; 400 r.p.m.; 2000 r.p.m. Arrows indicate the times for switching the potential to −0.5 V and to 0.0 V, respectively.

FIG. 18B shows chemiluminescence intensities as a function of ω^{1/2} (ω=rotation speed). In all experiments chemiluminescence was generated by the application of a potential step from E1=0.0 V to E2=−0.5 V and back vs. SCE. Data recorded in 0.01 M phosphate buffer, pH=7.4, that includes luminol, 1×10⁻⁶M, under air.

FIG. 19 shows the calibration curves corresponding to the chemiluminescence intensities upon analyzing extracts of 293-kidney cancer cells of a different number of cells, at constant rotation speeds of (i) 0 r.p.m., (ii) 60 r.p.m. and (iii) 2000 r.p.m. Inset: Enlargement of calibration curves showing the chemiluminescence signal intensities obtained from extracts containing 0-100 cells. The conditions for the recording of the chemiluminescence are as detailed in FIG. 18.

FIG. 20A Shows chemiluminescence intensities upon the analysis of a HeLa-cells extract containing 100,000 cells, at: 0 r.p.m.; 20 r.p.m.; 60 r.p.m.; 400 r.p.m.; 2000 r.p.m. Inset: Chemiluminescence intensities as a function of ω1/2 (ω=rotation speed). The conditions for the recording of the chemiluminescence are detailed in FIGS. 18A, B.

FIG. 20B depicts the calibration curves corresponding to the chemiluminescence intensities upon analyzing extracts containing a different number of HeLa cells at constant rotation speeds of 0 r.p.m., 60 r.p.m. and 2000 r.p.m. Inset: Enlargement of calibration curves showing the chemiluminescence signal intensities obtained from extracts containing: 0-100 cells.

FIG. 21 depicts electrogenerated chemiluminescence intensities obtained from extracts containing: (a) 1000 HeLa cells; (b) 1000 293-kidney cancer cells; (c) 100,000 NHF cells.

FIG. 22 shows electrogenerated chemiluminescence intensity obtained upon analyzing extracts from: (a) lung adenocarcinomas; (b) lung squamous epithelial carcinomas; (c) healthy tissues, (d) normal cells extract.

### DETAILED DESCRIPTION OF THE INVENTION

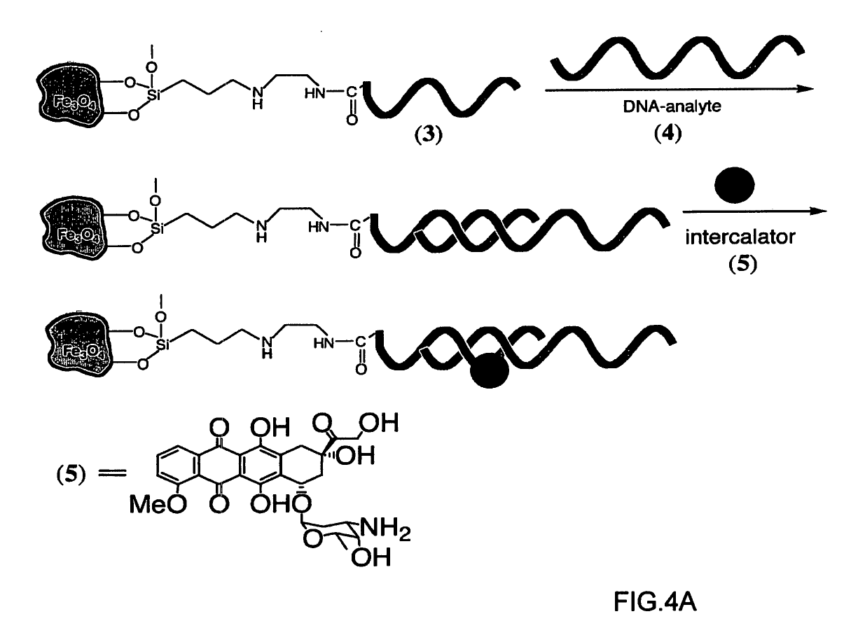

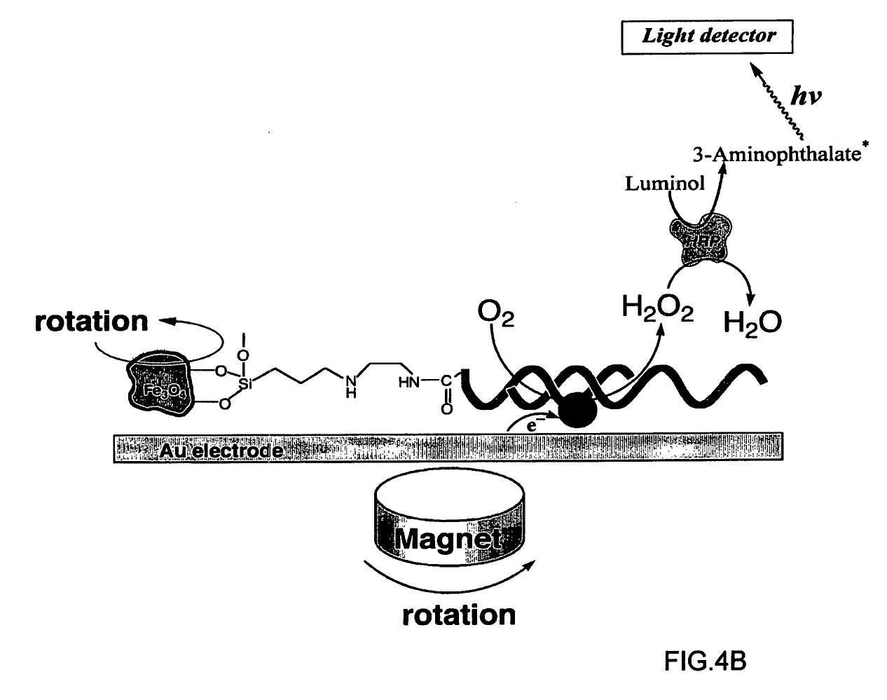

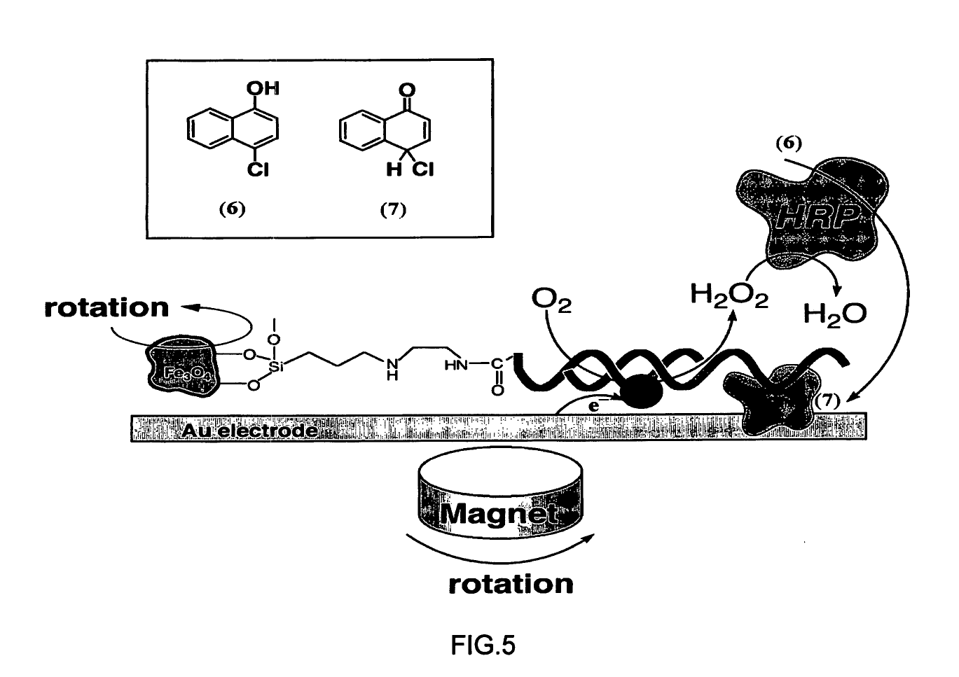

It should be noted that during an analysis performed by the method of the invention, at least one of the components of the chemical system, for example the analyte, the recognition moiety, the catalyst or a component needed for the catalyst's activity such as a substrate, should be dissolved in the analyzed liquid medium, whereas the other components are linked to the magnetic particles. In the examples below, the following components were in the respective solutions:
(a) for the NADH analysis—NADH was dissolved in the solution and PQQ was immobilized on the magnetic particles;
(b) for the analysis of glucose—glucose was dissolved in the solution together with glucose oxidase that functions as a biocatalyst, while ferrocene, which is the electron mediator providing electrical communication between the electrode and the enzyme, was immobilized at the magnetic particles;
(c) for the DNA analysis according to FIGS. 4A, 4B and 5—complementary DNA (the analyte) was immobilized on the DNA functionalized magnetic particles together with doxorubicin which functions as an electrocatalyst, while oxygen that is a substrate electrocatalytically converted into hydrogen peroxide, is soluble in the analyzed medium;
(d) antibody analysis—DNP-antibody is the analyte and was immobilized at the particle surface together with an electrocatalytic naphthoquinone.

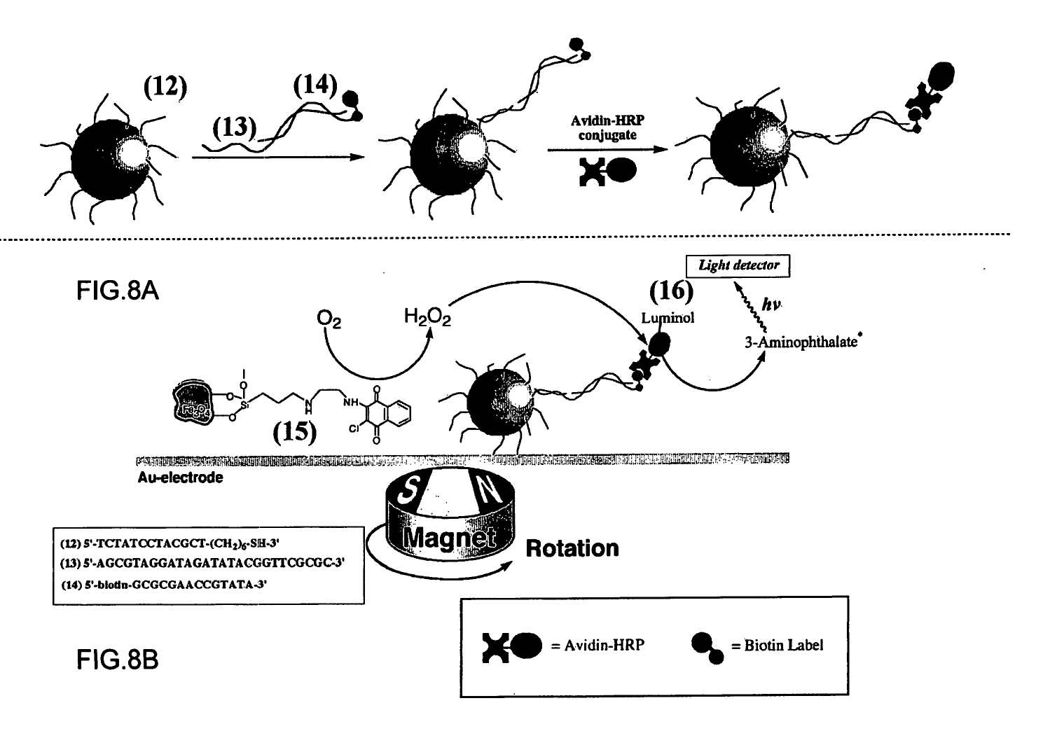

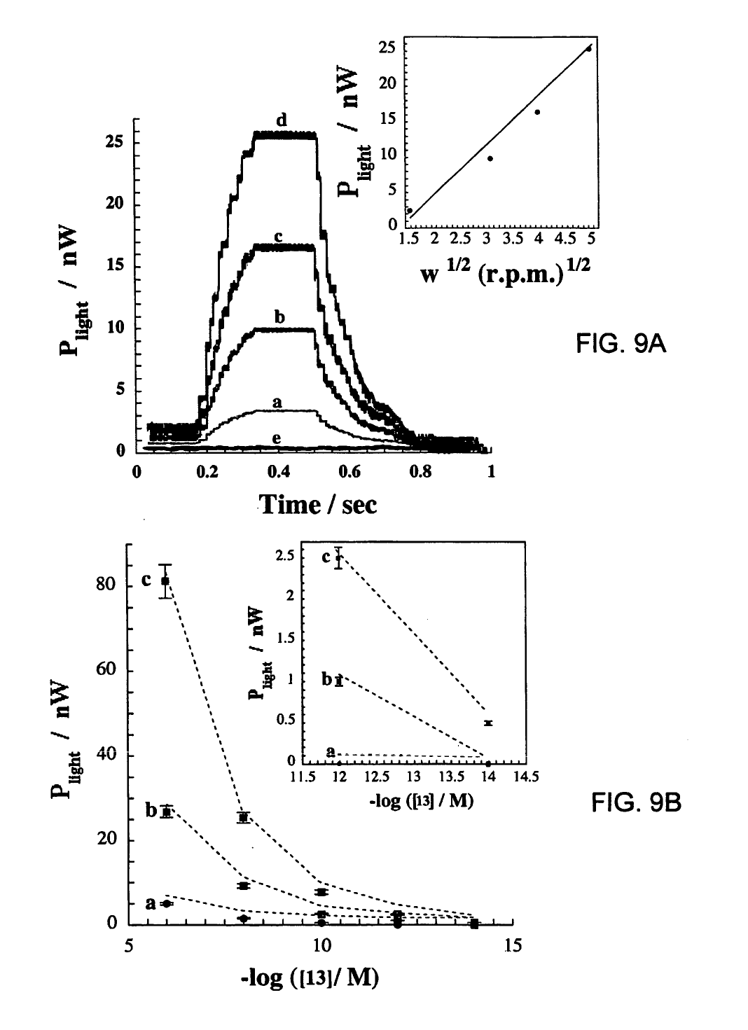

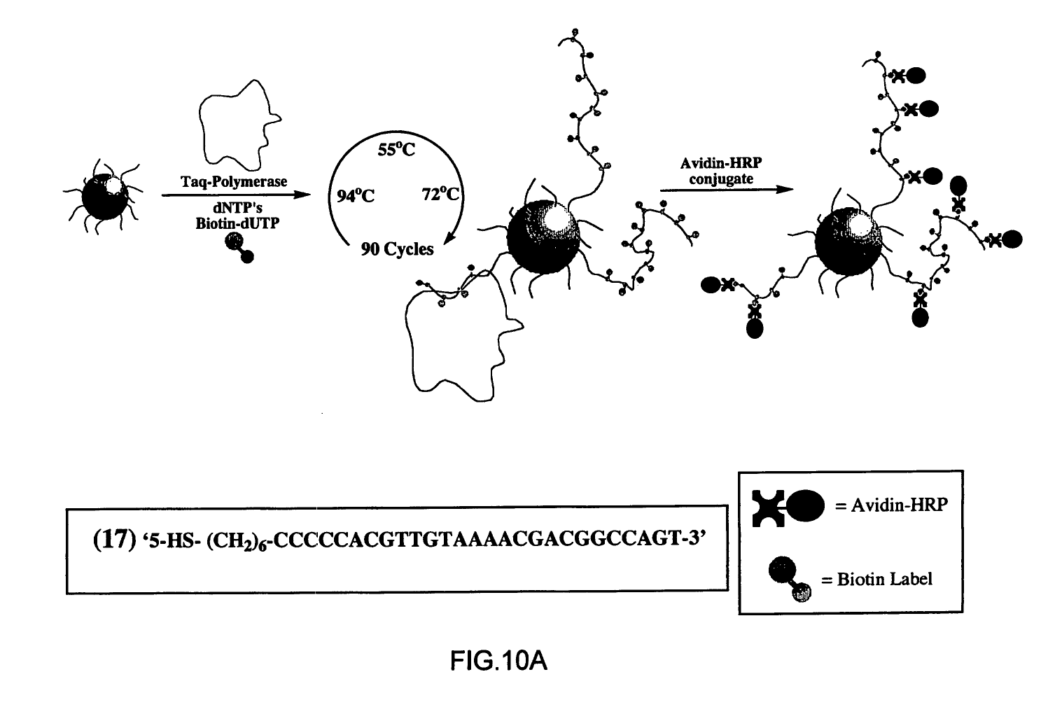

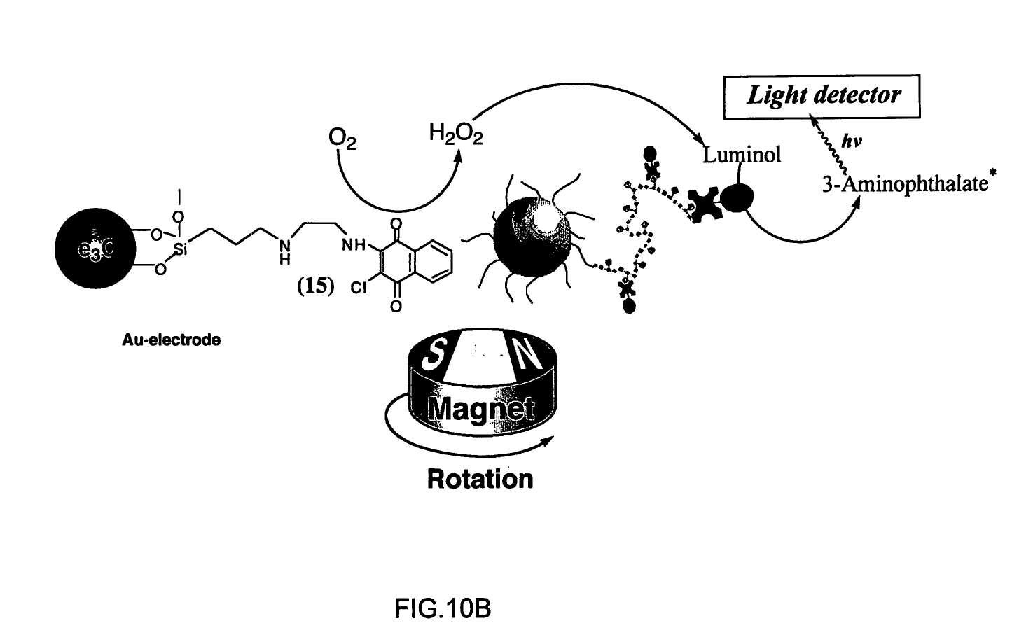

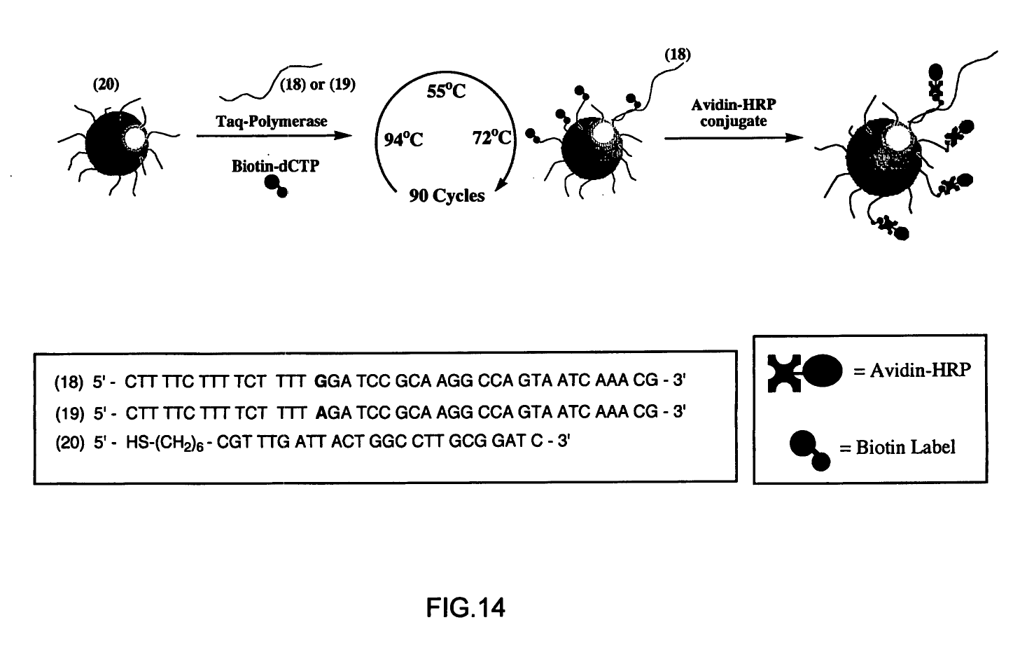

Oxygen is the solubilized material that is converted electrocatalytically to hydrogen peroxide.
(e) for the DNA analysis according to FIGS. 8A, 8B and 9—complementary DNA (the analyte) was immobilized on the DNA functionalized magnetic particles. An additional DNA reagent complementary to the analyte was immobilized to said complex, to which the enzyme horseradish peroxidase (HRP) was immobilized via a biotin-avidin interaction. Naphthoquinone was also immobilized to magnetic particles. Upon the application of a potential on the electrode, the naphthoquinone is reduced to hydroquinone and the electrocatalyzed reduction of oxygen to hydrogen peroxide occurs. The HRP-catalyzed oxidation of luminol by the electrogenerated hydrogen peroxide results in chemiluminescence and emission of light. Luminol and hydrogen peroxide are soluble in the analyzed medium;
(f) for the DNA analysis according to FIGS. 10-13—complementary DNA (M13 φ DNA; the analyte) was immobilized on the DNA functionalized magnetic particles and the complex was used as a substrate for Taq-Polymerase. The nucleotides (DATP, dCTP, dTTP and dGTP and biotin-dUTP) were soluble in the analyzed medium. After the polymerase reaction has been terminated, HRP was immobilized on the DNA linked magnetic particles via a biotin-avidin interaction. Electrocatalytic naphthoquinone was also immobilized to magnetic particles. Oxygen that is a substrate electrocatalytically converted into hydrogen peroxide, and luminol that, along with hydrogen peroxide is the substrate for HRP is also soluble in the analyzed medium;
(g) for the DNA analysis according to FIGS. 14-16—complementary DNAs (the mutant analyte and the wild-type DNA) were immobilized on the DNA linked magnetic particles and the complex was used as a substrate for Taq-Polymerase. The nucleotide, biotin-dCTP, was soluble in the analyzed medium. After the polymerase reaction has been terminated, HRP was immobilized on the DNA functionalized magnetic particles via a biotin-avidin interaction. Electrocatalytic naphthoquinone was also immobilized to magnetic particles. Oxygen that is a substrate electrocatalytically converted into hydrogen peroxide, and luminol that, along with hydrogen peroxide is the substrate for HRP is also soluble in the analyzed medium.

(h) For the telomerase analysis the enzyme analyte catalyzed the addition of telomeric repeats to the DNA primer, which was bound to the magnetic particles. The nucleotides (DATP, dCTP, dTTP and dGTP and biotin-dUTP) were soluble in the analyzed medium. After the telomerase reaction has been terminated, HRP was immobilized on the DNA linked magnetic particles via a biotin-avidin interaction. Electrocatalytic naphthoquinone was also immobilized to separate magnetic particles. Oxygen that is a substrate, was electrocatalytically converted into hydrogen peroxide, and luminol that, along with hydrogen peroxide is the substrate for HRP is also soluble in the analyzed medium.

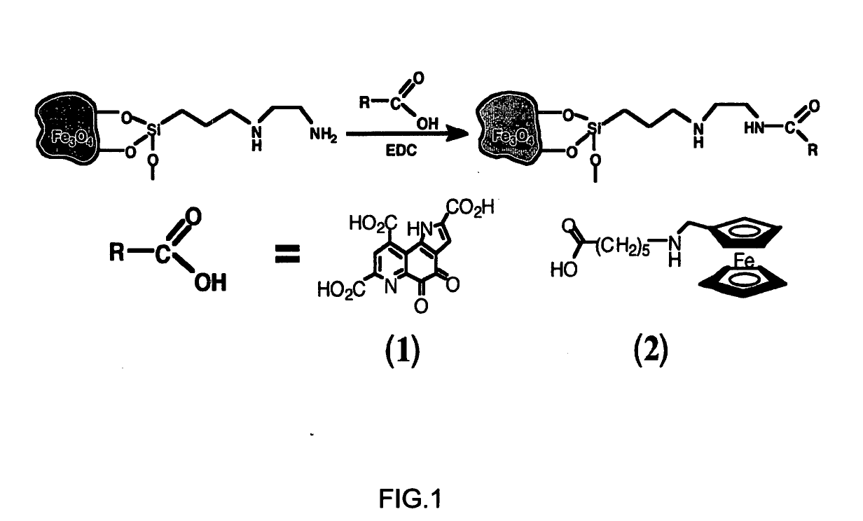

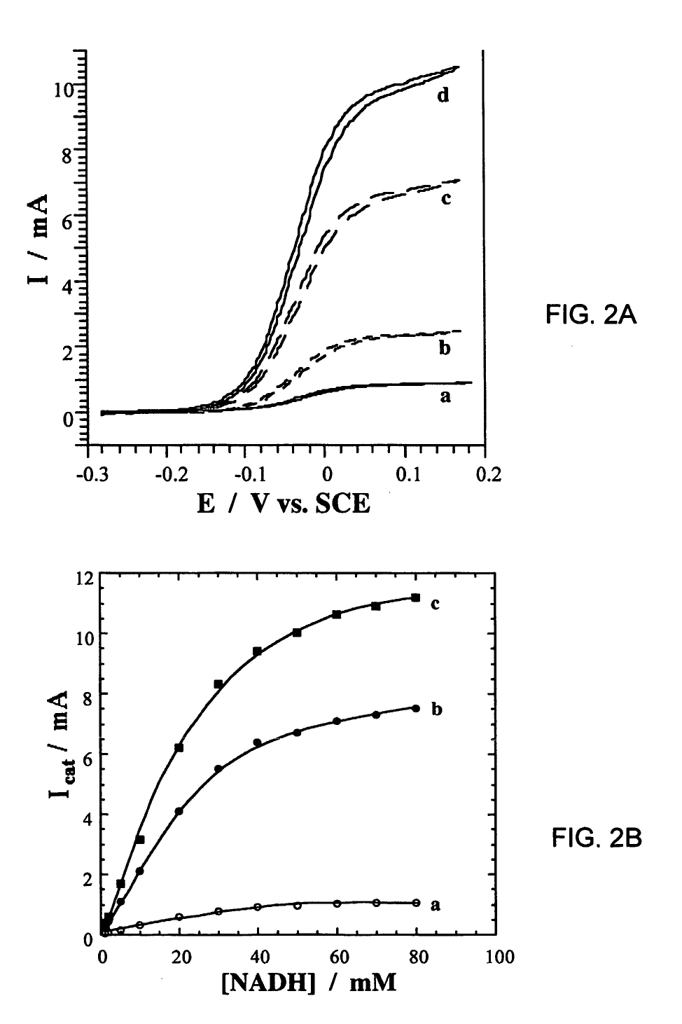

Magnetic particles (Fe₃O₄, ca. 1 μm average diameter, saturated magnetization ca. 65 emu·g⁻¹) were prepared according to the published procedure¹³ without including the surfactant into the reaction medium. FIG. 1 illustrates the functionalization of the magnetic particles. The magnetic particles were silanized with [3-(2-aminoethyl)aminopropyl]trimethoxysilane and then functionalized with pyrroloquinoline quinone, PQQ (1), or with N-(ferrocenylmethyl)aminohexanoic acid (2), using 1-ethyl-3-(3-dimethylaminopropyl)-carbodiimide, EDC, as a coupling reagent. The PQQ-functionalized magnetic particles attracted to a bottom Au-electrode (0.24 cm²) by the external magnet (NdFeB/Zn-coated magnet, 18 mm diameter, providing 0.2 kOe at the electrode surface), reveal a reversible cyclic voltammogram at E°=−0.13 V (vs. SCE), pH=7.0, indicating an average surface coverage of 7500 PQQ units per particle. The cyclic voltammogram of the PQQ units associated with the magnetic particles is independent of the rotation-speed of the external magnet, indicating that the redox-units are confined to the electrode support. The PQQ-functionalized magnetic particles act as an electrocatalyst for the oxidation of 1,4-dihydronicotineamide adenine dinucleotide, NADH, especially efficient in the presence of Ca²⁺ ions.¹⁴ FIG. 2A shows the cyclic voltammograms observed upon the PQQ-magnetite mediated electrocatalyzed oxidation of NADH, 50 mM, at different rotation-speeds of the external magnet. FIG. 2B shows the calibration curves corresponding to the anodic currents originating in the presence of different concentrations of NADH at variable rotation speeds of the external magnet. From FIGS. 2A and 2B it is evident that the resulting electrocatalytic currents increase as the rotation speed of the external magnet is elevated (the theoretical relation I_{cat} ∝ω^{1/2} is observed at low rotation speeds).

In a control experiment, PQQ-functionalized silica particles that gravimetrically settle on the Au-electrode, were subjected to different rotation-speeds of the external magnet in the presence of NADH. No effect of the external rotating magnet on the resulting electrocatalytic current was observed. This implies that the rotation of the magnetic particles on the electrode support leads to the increased electrocatalytic anodic currents upon rotation of the external magnet due to hydrodynamic control of the substrate mass-transport to the electrode.

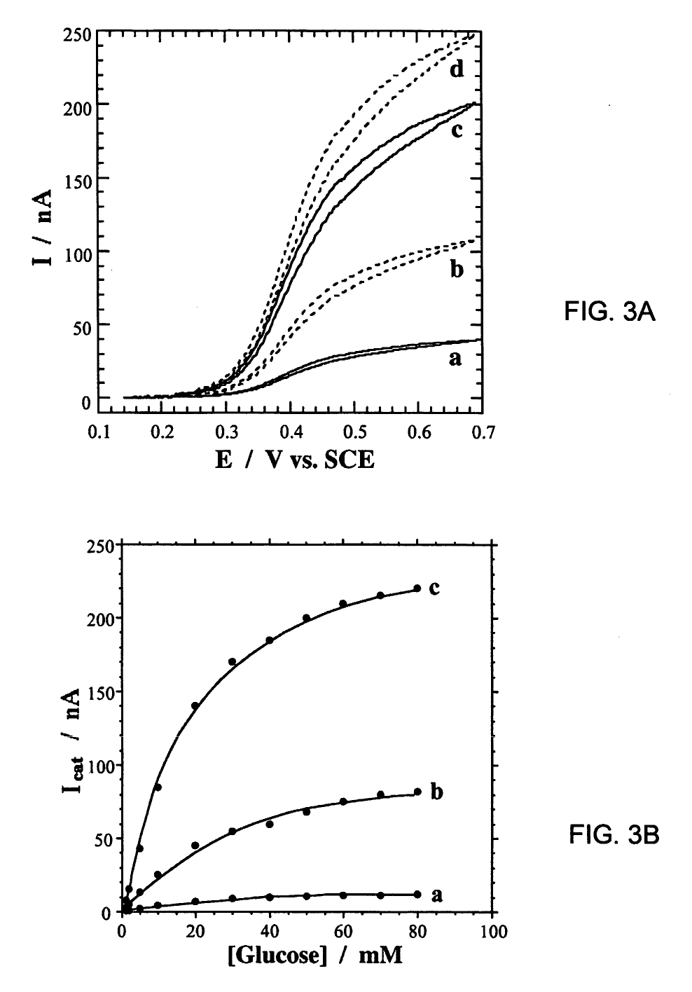

The magnetic-field stimulated enhancement of the electrocatalytic currents generated by the rotation of redox-functionalized magnetic particles was also demonstrated for bioelectrocatalytic transformations. The magnetic particles functionalized with the ferrocene derivative, (2), were attracted to the Au-electrode and rotated on the conducting support by means of the external rotating magnet. The quasi-reversible redox-wave of the ferrocene units, E°=0.32 V, is independent of the rotation of the external magnet. FIG. 3A shows the cyclic voltammograms of the ferrocene-functionalized magnetic particles in the presence of glucose oxidase (GOx), 1×10⁻⁵ M, and glucose, 50 mM, at different rotation rates of the external magnet. FIG. 3B shows the calibration curves corresponding to the amperometric responses of the system at different concentrations of glucose and variable speeds of rotation of the external magnet. The electrocatalytic anodic currents increase as the external rotation speed of the magnet is elevated. Control experiments reveal that the electrocatalytic anodic currents are observed only in the presence of glucose oxidase and glucose, and that no effect of the rotation speed of the external magnet on the electrocatalytic anodic currents generated by ferrocene-functionalized SiO₂ particles is observed.

Another experiment, as illustrated in FIGS. 4A and 4B, shows DNA analysis using bioelectrocatalytic light emission. The Fe₃O₄ magnetic particles were silanized and a DNA primer (3) was covalently linked to the silane thin film. The DNA functionalized magnetic particles were reacted with the DNA-analyte (4) resulting in a double stranded (ds) DNA helix. The ds-DNA functionalized magnetic particles were reacted with a doxorubicin intercalator (5) that binds specifically to the ds-DNA. This intercalator is an electrochemically active quinone that can be reduced electrochemically and can further reduce O₂, resulting in production of hydrogen peroxide, H₂O₂. The electrocatalytically produced H₂O₂, in the presence of horseradish peroxidase (HRP) and luminol, generates light emission. The emitted light is an analytical signal reporting on the presence of the intercalator and thus on the presence of the analyte DNA (4). The light emission intensity depends on the rate of the electrocatalytic reduction of O₂. This rate is enhanced upon the rotation of the modified magnetic particles, thus resulting in the amplification of the light emission.

In another experiment, DNA analysis was carried out using bioelectrocatalytic precipitation of an insoluble material. The system, illustrated in FIG. 5, is similar to the one described above, but the electrocatalytically generated H₂O₂ in the presence of HRP and 4-chloronaphthol (6) results in the precipitation of the insoluble product (7). The insoluble product isolates the electrode surface. This effect can be measured by means of Faradaic impedance or chronopotentiometry. The extent of the electrode isolation depends on the rate of H₂O₂ production. This rate is enhanced by the rotation of the modified magnetic particles, thus resulting in the amplification of the signal.

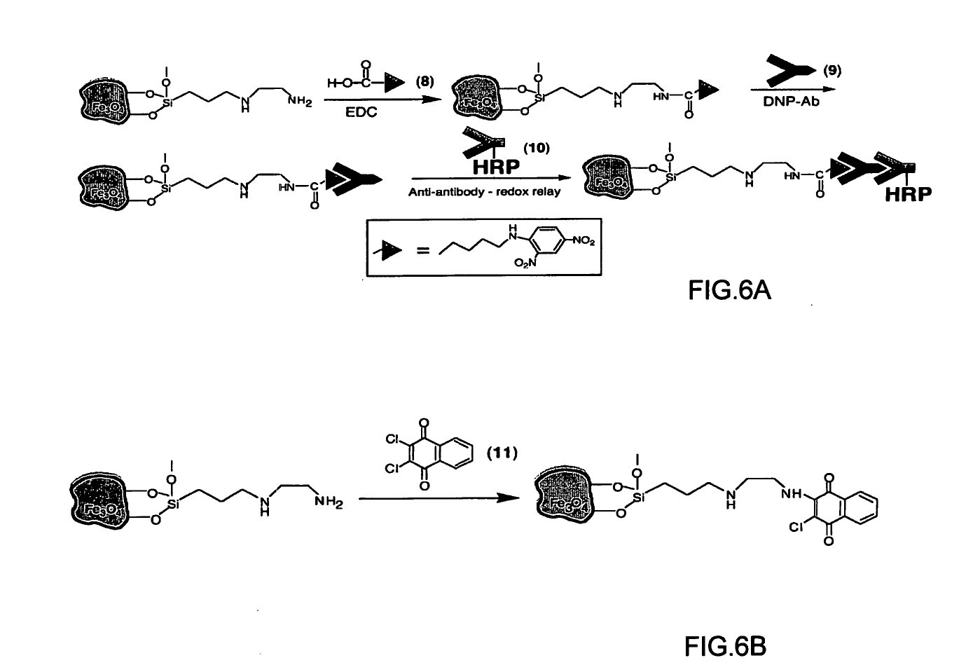

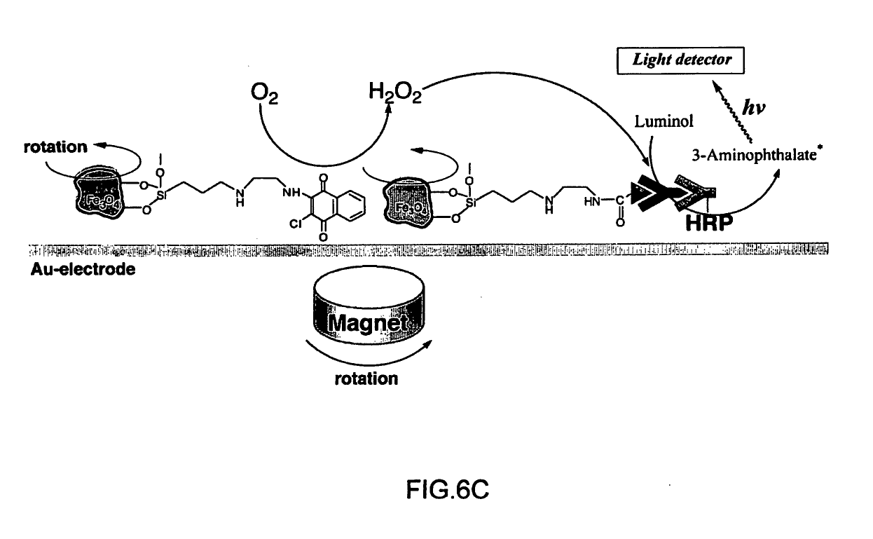

A new immunosensor is illustrated in FIGS. 6A-C. Magnetic particles were silanized with aminosilan as described above. An antigen that is a carboxylic derivative of dinitrophenyl, (8), is covalently coupled to the amino groups of the siloxane layer at the surface of magnetic particles. The coupling reaction with the silanized magnetic particles, 10 mg, proceeds with (8) at a concentration of 1 mM in the presence of EDC, 5 mM, in 0.1 M HEPES buffer, pH 7.2, for 2 hours. Then the (8)-derivatized magnetic particles were washed with water in order to remove all unbound antigen molecules. The antigen modified magnetic particles are reacted with various concentrations of DNP-antibody, (9) (DNP being the abbreviation of dinitrophenol), (from 2 ng per mL to 50 ng per mL) in 0.1 M phosphate buffer, pH 7.0, for 30 minutes. Then the antibody/antigen-functionalized magnetic particles are reacted with anti-DNP-antibody conjugated with the enzyme horseradish peroxidase (HRP), (10), 100 ng per mL, for 30 minutes. This secondary anti-DNP-antibody, (10), is capable of binding to the primary DNP-antibody, but not to the DNP-antigen (8). Thus, the amount of the HRP-conjugate-anti-DNP-antibody, (10), bound to the magnetic particles is dependent on the presence of the DNP-antibody and it is proportional to the later concentration. The described procedure of the magnetic particles functionalization with the antigen, (8), the DNP-antibody, (9), and the HRP-conjugate-anti-DNP-antibody, (10), is shown in FIG. 6A. The enzyme HRP uses hydrogen peroxide (H₂O₂) and luminol to produce light. Thus, H₂O₂ may be introduced to the sample as part of the assay solution. However, in this example, another kind of functionalized magnetic particles is used to produce H₂O₂ and thus activate the system electrochemically, as illustrated in FIG. 6B. The silanized magnetic particles are reacted with 2,3-dichloro-1,4-naphthoquinone, (11), in ethanolic suspension (5 mL) containing 10 mg of magnetic particles and 100 mg of the quinone (11) for 3 minutes upon boiling the ethanolic suspension. Then the quinone derivatized magnetic particles are washed 3 times with ethanol and once with water. A system implementing the functionalized particles illustrated in both FIGS. 6A and 6B is illustrated in FIG. 6C. The system is composed of 10 mg of the quinone (11)-functionalized magnetic particles produced according to FIGS. 6B and 10 mg of the magnetic particles created according to FIG. 6A. The system also includes an Au-plate electrode and the rotating magnet below the electrode. The solution also includes luminol, 1×10⁻⁵ M, in 0.1 M phosphate buffer, pH 7.0, saturated with air. A light detector is fixed above the solution. A potential of −0.6 V (vs. SCE) is applied to the electrode, that provides electrochemical reduction of oxygen dissolved in the solution. This reduction is catalysed by the quinone (11) and results in the formation of hydrogen peroxide (H₂O₂). The hydrogen peroxide reacts with luminol in the presence of the enzyme HRP resulting in the light emission detected by a light detector.

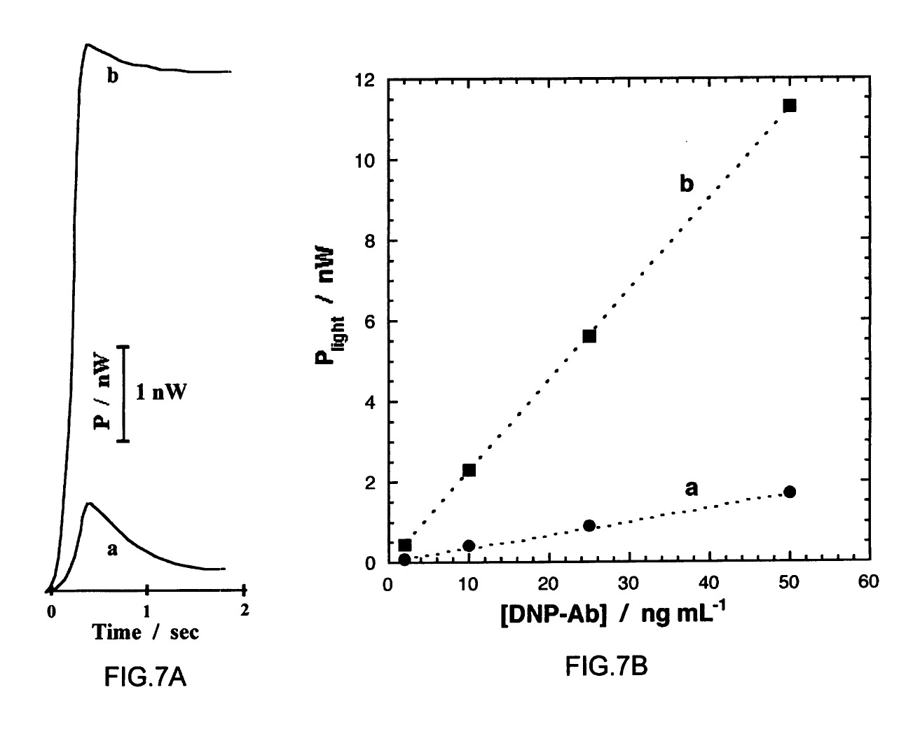

FIGS. 7A and 7B show the results of the immunosensing system illustrated in FIG. 6C. FIG. 7A shows the intensities of the light emission without rotation of the magnetic particles (curve a) and with 100 rpm (rotations per minute) (curve b). The signal is amplified because of the enhanced mass transport in the system upon the rotation (transport of oxygen to the quinone, hydrogen peroxide from the quinone to the HRP, and luminol to the HRP). The light emission depends on the amount of the bound enzyme HRP, but its concentration is dependent on the concentration of the DNP-antibody (9) (analyte). FIG. 7B shows two calibration plots (the light signal intensity vs. the DNP-antibody concentration): without rotation (a) and with 100 rpm (b). The ratio between the corresponding experimental points on curves (b) and (a) presents the amplification factor achieved upon the rotation of the magnetic particles. It should be noted that the amplification factor is dependent on the rotation speed (however, the dependence is not linear—see the previous examples).

FIG. 8A illustrates an embodiment of the invention providing the detection of a DNA analyte by use of DNA-functionalized magnetic particles, DNA labeled with biotin and an avidin-HRP. Amine-functionalized borosilicate-based magnetic particles (5 μm, MPG® Long Chain Alkylamine, CPG Inc.) were modified with a DNA primer (12) using the heterobifunctional crosslinker 3-maleimidopropionic acid N-hydroxysuccinimide ester. The coverage of the particles was estimated using the Oligreen® reagent (ssDNA Quantitation Assay Kit Molecular Probes, Inc.) to be ca. 52,000 oligonucleotide molecules-particle⁻¹. The primer (12) is complementary to a part of the target sequence (13). The (12)-functionalized magnetic particles are hybridized in a single step with a mixture that includes (variable concentrations) the target (13) and the biotin-labeled nucleic acid, (14), that is complementary to the free segment (13). The three-component double-stranded DNA assembly (12)/(13)/(14) is then interacted with avidin-horseradish peroxidase (HRP) that acts as a biocatalytic label.

According to one embodiment, depicted in FIG. 8B the DNA/avidin-HRP functionalized magnetite particles of FIG. 8A are subsequently mixed with magnetite particles modified with the naphthoquinone unit (15). The mixture of the magnetic particles is then attracted to an electrode support by means of an external magnet. Electrochemical reduction of the naphthoquinone to the respective hydroquinone results in the catalyzed reduction of O₂ to H₂O₂. The electrogenerated H₂O₂ leads, in the presence of luminol, (16), and the HRP enzyme label to the generation of the chemiluminescence signal.

The avidin-HRP approaches the electrode only if the target DNA hybridizes with the magnetic particles, provided that non-specific adsorption does not take place. Thus, chemiluminescence occurs only if the target DNA (13), is in the analyzed sample. Furthermore, the light intensity relates directly to the number of recognition pairs of (12) and (13) associated with the electrode, and thus it provides a quantitative measure to the concentration of (13) in the sample.

The rotation of the particles on the barrier surface by means of the rotating external magnet results in the enhanced electrogenerated chemiluminescence, since the magnetic particles behave as rotating microelectrodes, where the interaction of O₂ and luminol with the catalysts on the electrode is controlled by convection rather than by diffusion. Thus, the rotation of the magnetic particles is anticipated to yield the amplified detection of DNA.

It should be appreciated that electrogeneration of H₂O₂ is not a necessary part of the invention, and according to a different embodiment, H₂O₂ may be directly introduced to the assay sample. In such case, the electrode is also not necessary. However, in such alternative embodiment, as H₂O₂ is not localized near an electrode, excess avidin-HRP must be removed from the assayed sample prior to providing the reaction conditions.

In an experiment carried out essentially according to FIGS. 8A and 8B, a sample of (12)-functionalized magnetic particle was interacted with (13), 1.4×10⁻⁸ M, in the presence of the biotinylated nucleic acid, (14), 2×10⁷ M. The resulting double-stranded (12)/(13)/(14) tri-component system was collected by the external magnet, washed with 0.2 M phosphate buffer (pH 7.4), and then reacted with the avidin-HRP conjugate and again collected by the external magnet. The resulting particles were suspended in the electrochemical cell together with the naphthoquinone (15)-modified magnetic particles, 2 mg·ml⁻¹. In FIG. 9A, curve (a) shows the emitted light intensity upon the collection of the magnetic particles on the electrode by means of the external magnet, and the application of a potential step on the electrode from 0 V to −0.5 V and back. FIG. 9A, curve (b)-(d) shows the emitted light intensities upon the rotation of the particles by means of the external magnet, using different rotation speeds. Increase of the rotation speed enhances the intensity of the emitted light, and the resulting light intensity relates linearly to ω^{1/2} (ω=rotation speed), as expected for electrocatalytic rotating microelectrodes. In a control experiment that lacks (13) in the hybridization step, no light emission is detected, indicating that no non-specific adsorption of (14) or the avidin-HRP conjugate takes place. The light intensity emitted from the system relates to the surface coverage of the avidin-HRP conjugate, and this is controlled by the amount of (13)/(14) associated with the particles and thus determined by the concentration of (13). FIG. 9B, shows the derived calibration curves corresponding to the emitted light intensities upon analyzing different concentrations of (13) and recorded at different rotation speeds. FIG. 9A, curve (e), shows the light intensity observed upon the analysis of the mutant (13a), that includes a 7-base mutation sequence in respect to (13), 1×10⁻⁷ M, at a rotation speed of 2000 r.p.m. according to the embodiment of FIGS. 8A and 8B. No emitted light due to non-specific adsorption of the avidin-HRP conjugate on the surface, is observed. This light intensity is considered as the background signal, and thus (13) can be sensed in this example with a detection limit of 1×10⁻¹⁴ M at ω=2000 r.p.m. (S/N>3).

The following examples show use of the detection of a DNA analyte according to this invention where the reaction signal is amplified using polymerase chain reaction. In those examples, the following experimental conditions and materials were used:
Amine-functionalized borosilicate-based magnetic particles (5 μm, MPG® Long Chain Alkylamine, CPG Inc.), Biotin-21-dUTP (Clontech). The heterobifunctional crosslinker 3-maleimidopropionic acid N-hydroxysuccinimide ester, oligonucleotides (17), (18), (19) and (20), Avidin-HRP conjugate, dNTP's, Biotin-11-dCTP, Taq Polymerase, 10×PCR buffer and all other compounds were purchased from Sigrna and used as received.
Preparation of DNA-functionalized magnetic particles: 30 mg of the amino-functionalized magnetic particles (MPG® Long Chain Alkylamine, CPG Inc.) were activated by reaction with the heterobifunctional crosslinker 3-maleimidopropionic acid N-hydroxysuccinimide ester (10 mg, Sigma) in 1 ml of DMSO. After 4 hrs of incubation at room temperature, the particles were collected with and external magnet and thoroughly washed with DMSO and water. The maleimido-activated particles were then reacted with 20-30 O.D. of the thiolated oligonucleotide in phosphate buffer 0.1M, pH 7.4 for a period of 8 hrs. (The thiolated nucleotide was freshly reduced with DTT and separated on a Sephadex G-25 column prior to the reaction with the functionalized particles). Finally, the magnetic particles were washed with water and phosphate buffer 0.1M, pH7.4. In order to keep the DNA-modified particles for periods longer than one week, 1% w/v sodium azide was added, and the particles were kept at 4° C. The oligonucleotide content on the magnetic particles, before and after enzymatic DNase treatment (10 units DNase, 30 min at 37° C.) was measured by the use of the Oligreen® reagent (ssDNA Quantitation Assay Kit Molecular Probes, Inc.).
φ: (a) For single-point-mutation detection: denaturation 30 sec, 94° C.; annealing 30 sec, 55° C.; polymerization 5 sec, 72° C. (b) For Viral detection: denaturation 30 sec, 94° C.; annealing 30 sec, 55° C.; polymerization 15 sec, 72° C.
An Au-coated (50 nm gold layer) glass plate (Analytical-μSystem, Germany) was used as a working electrode (0.3 cm² area exposed to the solution). An auxiliary Pt electrode and a quasi-reference Ag electrode were made from wires of 0.5 mm diameter and added to the cell. The quasi-reference electrode was calibrated vs. saturated calomel electrode and the potentials are given vs. SCE. An open electrochemical cell (230 μL) that includes the Au-electrode in a horizontal position and a light detector linked to a fiber optics enabled easy light emission measurements upon application of the appropriate potential to the modified working electrode. The electrochemical measurements were performed using a potentiostat (EG&G, model 283) connected to a computer (EG&G Software 270/250 for). All the measurements were performed in 0.01 M phosphate buffer solution, pH 7.0, at room temperature. The electrochemically-induced chemiluminescence was measured with a light detector (Laserstat, Ophir) linked to an oscilloscope (Tektronix TDS 220). The light detector was connected to the electrochemical cell by an optical fiber. The background electrolyte solution was equilibrated with air and included luminol, 1×10⁻⁶ M.

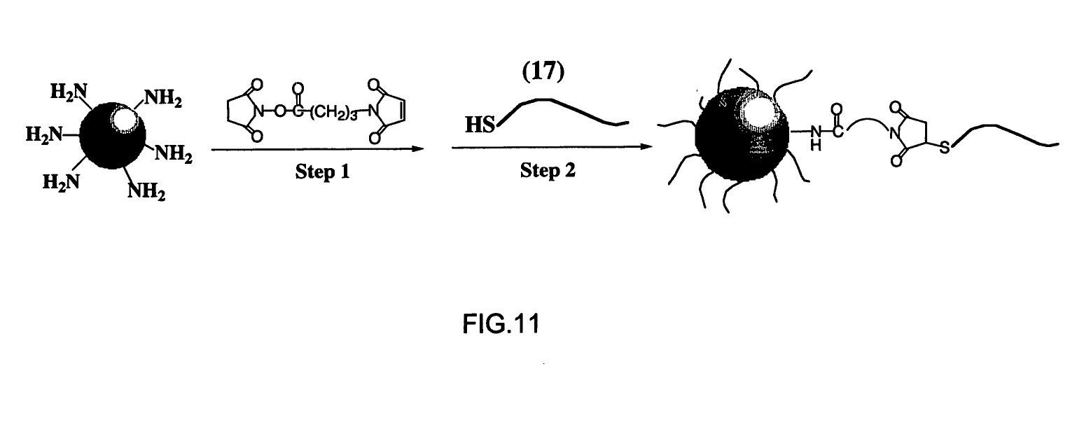

FIG. 10 depicts a method for the amplified detection of the viral MP13φ DNA, using functionalized magnetic particles. Magnetic particles (MPG® Long Chain Alkylamine, 5 μm diameter, CPG Inc.) are modified with the thiolated primer (17) using the heterobifunctional cross-linker 3-maleimidopropionic acid N-hydroxysuccinimide ester, as outlined in FIG. 11. The average coverage of the magnetite particles was determined by using the Oligreen® reagent (Molecular Probes, Inc.) and corresponds to ca. 50,000 oligonucleotide units-particle⁻¹. The number of nucleic acids that are associated with the particles and accessible to an external enzyme was estimated by subjecting the (17)-functionalized magnetic particles to DNase and by the subsequent determination of the content of the DNA that is cleaved off. It was found that ca. 20,000 oligo units-particle⁻¹ are cleaved off, implying that only ca. 40% of the particle-linked nucleic acids are accessible to the enzyme. As shown in FIG. 10, The (17)-modified magnetic particles are hybridized with the MP13φ DNA (7229 bases) and are subjected to polymerization in the presence of a mixture of dGTP; dATP; dCTP and biotinylated dUTP (b-dUTP). The polymerization introduces into the replica a high number of biotin labels. The replication is followed by thermal cycles that result in the dissociation of the analyzed MP13φ DNA, its re-hybridization with other oligonucleotide primers associated with the magnetic particles, and the subsequent polymerization and formation of new replica containing a high number of biotin label units. By controlling the number of thermal cycles, the replication on the particles' surface yields very high densities of biotin-labeled nucleic acids on the magnetic particles. The thermal cycles were conducted for 30 sec each, which translate to a replication efficiency of ca. 500-bases per cycle. This relatively low replication efficiency was purposely designed in order to eliminate steric crowding by the nucleic acid replica on the magnetite particles that might perturb the cyclic labeling of the particle by the biotin labels. The resulting biotin-labeled magnetic particles are then separated by means of the external magnet, reacted with avidin-horseradish peroxidase (HRP), and again separated by the external magnet and washed with a phosphate buffer solution. A mixture of the avidin-functionalized magnetic particles, 1 mg, and naphthoquinone-modified magnetic particles (15) (not shown) is then introduced into the electrochemical cell. The magnetic particles are then attracted to the electrode by means of the external magnet. Upon the application of a potential on the electrode that reduces the naphthoquinone to the respective hydroquinone, the electrocatalyzed reduction of O₂ to H₂O₂ occurs. The HRP-catalyzed oxidation of luminol, by the electrogenerated H₂O₂ results in chemiluminescence and the emission of light.

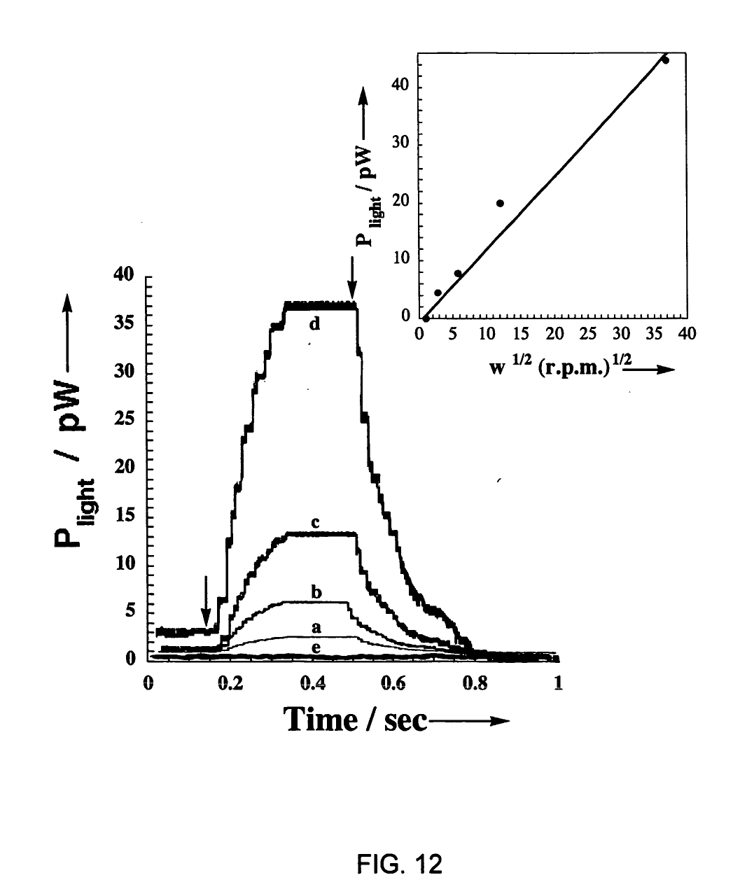

FIG. 12, curve (a), shows the emitted light intensity upon analyzing MP13 φDNA, 8.3×10⁻⁹M, according to the above, and by the application of a potential step on the electrode from 0 V to −0.5 V and back (the E° for the naphthoquinone-modified particles at pH=7 is 0.4 V). In a control experiment in which all the analysis steps were applied on a sample that lacks MP13φ? DNA, (curve (e)) no light emission was observed, indicating that no non-specific adsorption of the avidin-HRP conjugate on the electrode, or on the magnetic particles, takes place, FIG. 12.

The effect of rotation of the magnetic particles by means of the rotating external magnet is depicted in FIG. 12, curves (b)-(d). As the rotation speed of the magnetic particles is elevated, the emitted light intensity increases, and a linear relationship between the intensity of emitted light and ω^{1/2} (ω=the rotation speed) is observed, FIG. 12 (inset).

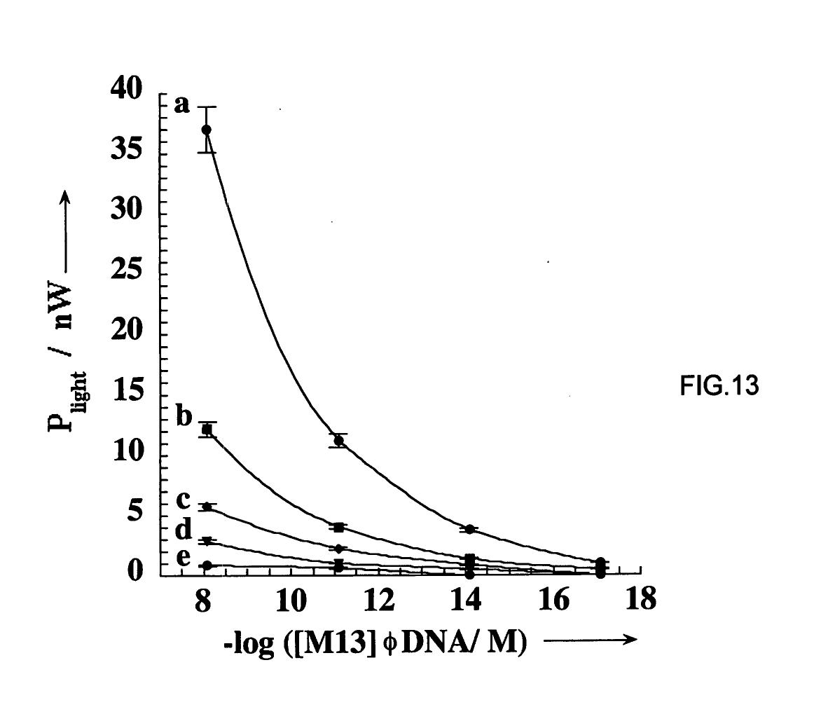

At a constant rotation speed of the particles, the intensity of emitted light is controlled by the surface coverage of the labeled nucleic acid associated with the magnetic particles, and this relates to the concentration of MP13φ? DNA in the analyzed sample during the replication cycles. FIG. 13 shows the emitted light intensity upon the analysis of different concentrations of MP13φ? DNA at a rotation speed of ω=2000 r.p.m.

A further example of the invention employs functional magnetic particles for the amplified detection of single base mismatches in DNA. This is exemplified by the analysis of the mutant sequence (18), where a G-base exchanges the A-base in the normal sequence gene (19), as shown in FIG. 14. The magnetic particles are functionalized with the nucleic acid (20) that is complementary to the mutant sequence, (18), and the normal gene sequence (19), up to one base prior to the mutation site. Interaction of the (20)-modified magnetic particles with the samples that include either (18) or (19) results in their hybridization with the particles. Treatment of the hybridized assemblies associated with the magnetic particles with polymerase and biotinylated-dCTP, followed by the application of thermal dissociation/annealing/labeling cycles results in the multi-labeling of the magnetic particles with biotin units upon the analysis of (18), whereas no biotin labels are introduced upon the analysis of (19). The subsequent interaction of the particles with the avidin-HRP conjugate, followed by the separation of the particles by means of the external magnet yield the biocatalytically labeled particles. Mixing of the resulting particles with the naphthoquinone-modified particles (15)(not shown) in the electrochemical cell, followed by their attraction to the electrode by means of the external magnet leads to the electrocatalyzed reduction of O₂ to H₂O₂, and in the presence of luminol, to the emission of light upon the analysis of (18), while no light is detected upon the analysis of (19). Rotation of the magnetic particles by means of an external magnet is then expected to amplify the emitted light since the electrogenerated chemiluminescence is controlled by convection of the respective substrates to the particles.

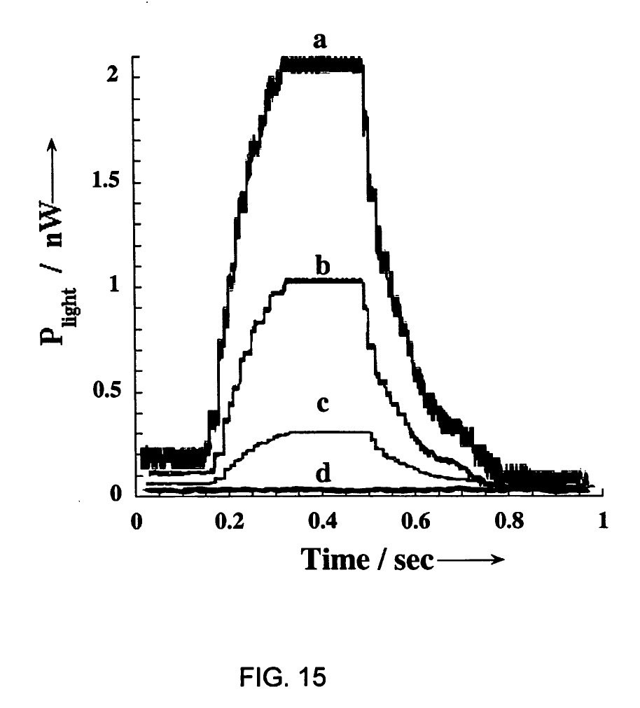

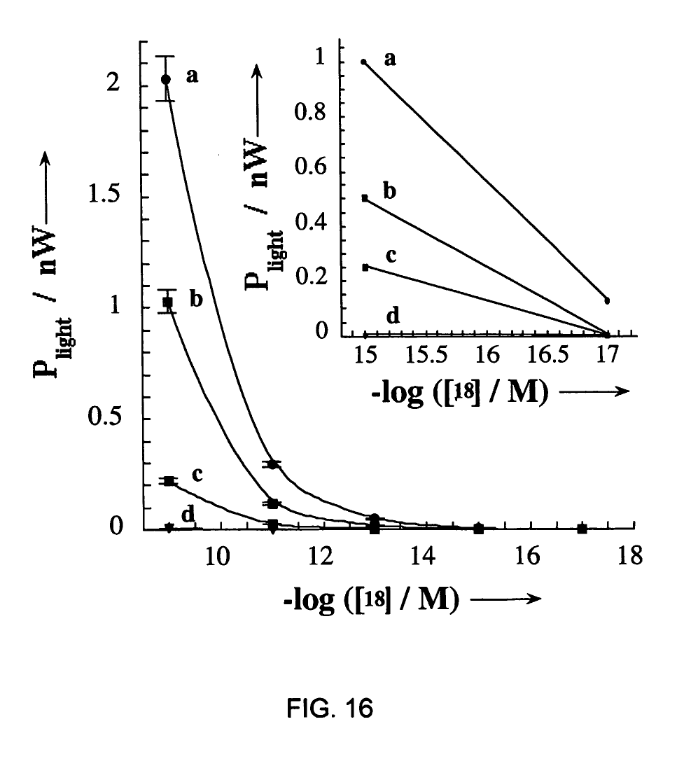

FIG. 15 shows the emitted light intensity at a rotation speed of 2000 r.p.m., upon analyzing (18), 1×10⁻⁹M, curve (a), and (19), 1.4×10⁻⁶M, curve (d), all according to FIG. 14. A potential step from 0 V to −0.5 V and back is applied on the electrode in order to activate the electrocatalyzed reduction of O₂, and to drive the secondary chemiluminescent process. No light emission is observed upon the analysis of (19), indicating that no biotin labels were incorporated into the nucleic acid-modified magnetic particles. Clearly, light emission is observed only upon the analysis of the mutant sequence. FIG. 15, curves (a)-(c), shows the light emitted from the system upon the rotation of the magnetic particles at different rotation speeds. FIG. 16 shows the light emitted upon analyzing different concentrations of (18), at different rotation speeds. The intensity of the emitted light increases as the rotation speed of the particles is elevated, (P ∝ω^{1/2}), implying that the processes at the electrode support are controlled by convection. The mutant sequence (18) is analyzed with a detection limit of 1×10⁻¹⁷ M. The concentration of (19) in the analyzed sample is 10³-fold higher than that of (18), and still no light emission is observed upon analyzing (19). This implies that no non-specific association of the HRP conjugate to the magnetic particles occurs.

In conclusion, this example described a magnetically amplified DNA analysis process. Several consecutive steps in the process lead to the overall amplification: (i) The thermal cyclic replication of the analyte on the magnetic particles leads to the incorporation of a high number of label-units into the nucleic acids linked to the particles. (ii) The electrocatalytic generation of O₂ at the electrode, and the coupled biocatalyzed light emission yield numerous product molecules or photons as a result of a single recognition event. (iii) The rotation of the magnetic particles leads to the amplified light emission since the transport of the substrates for the electrocatalytic and biocatalytic processes at the particles are convection-controlled. Using these methods, very high sensitivities were achieved.

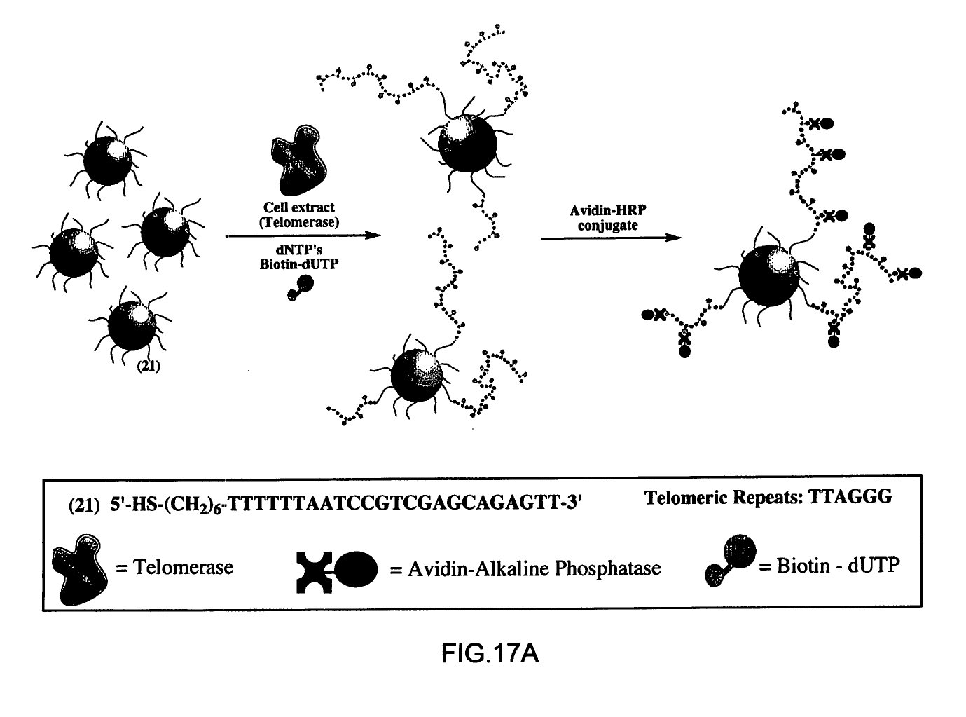

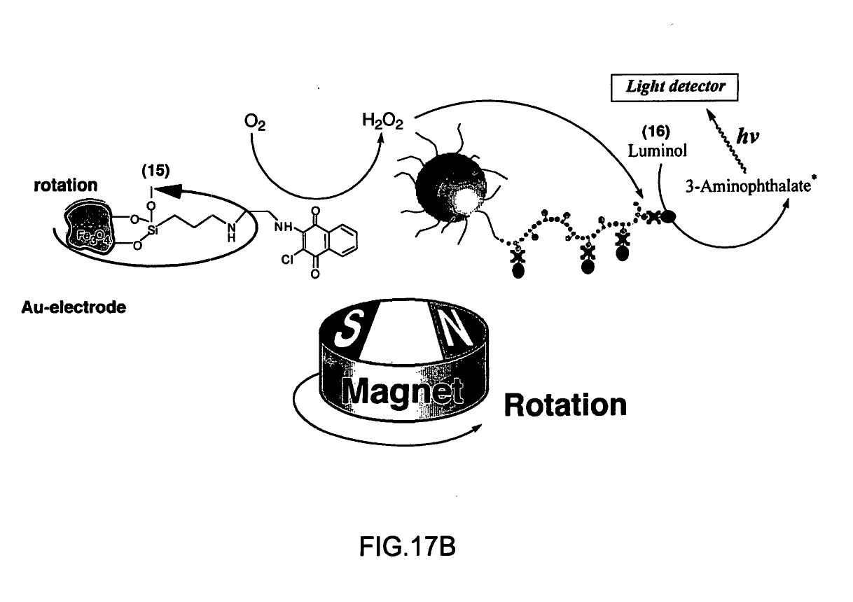

Yet another example of the invention is the detection of an enzyme in a given sample. Such detection of the enzyme analyte telomerase is schematically depicted in FIG. 17A. The analyte, telomerase, is a ribonucleoprotein complex capable of synthesizing new telomers by the addition of telomeric repeats to the 3′-end of chromosomal DNA. Accordingly, the recognition agent in this example is a nucleic acid sequence (21) that comprises a 6 T-base linker unit followed by a characteristic sequence recognized by the telomerase. Amine-functionalized magnetic particles (5 μm diameter) were activated with the bifunctional reagent 3-maleimidopropionic acid-N— hydroxysuccinimide ester, substantially as outlined in FIG. 11 (this time with sequence (21) instead of sequence (17)). The mercaptohexyl-modified nucleic acid, (21), was covalently linked to the magnetic particles. Specifically, 30 mg of the amino-functionalized magnetic particles (MPG® Long Chain Alkylamine, CPG Inc.) were activated by reaction with the heterobifunctional crosslinker 3-maleimidopropionic acid N-hydroxysuccinimide ester (10 mg, Sigma) in 1 mL of DMSO. After 4 hrs of incubation at room temperature, the particles were collected with an external magnet and thoroughly washed with DMSO and water. The maleimido-activated particles were then reacted with 20-30 O.D. of the thiolated oligonucleotide in phosphate buffer 0.1 M, pH 7.4 for a period of 8 hrs. (The thiolated nucleotide was freshly reduced with DTT and separated on a Sephadex G-25 column prior to the reaction with the functionalized particles). Finally, the magnetic particles were washed with water and phosphate buffer, 0.1 M, pH 7.4. In order to keep the DNA-modified particles for periods longer than one week, 1% w/v sodium azide was added, and the particles were kept at 4° C. The oligonucleotide content on the magnetic particles, before and after enzymatic DNase treatment (10 units DNase, 30 min at 37° C.) was measured by the use of the Oligreen® reagent (ssDNA Quantitation Assay Kit Molecular Probes, Inc.)

As schematically shown in FIG. 17A, the functional magnetic particles comprising the recognition agent (21) are treated with cell extract (which is assayed for the presence of the analyte) in the presence of a mixture of nucleotides dNTP that includes biotin labeled dUTP. The association of telomerase to the recognition agent is followed by telomerization that involves the labeling of the newly synthesized chains with biotin integrated into the telomeric repeats. The subsequent binding of avidin-horseradish peroxidase (HRP) introduces the biocatalytic labels into the telomer chains. The magnetic particles are collected by attraction to the bottom of the analyzing flask by means of an external magnet and washed to remove any residual cell extract or non-specifically absorbed HRP conjugate.

As schematically shown in FIG. 17B the resulting particles of FIG. 17A are then mixed with the naphthoquinone-functionalized magnetite particles (15) synthesized by the reaction of 3,4-dichloronaphthoquinone with aminoethylamine-modified magnetic particles. The mixture of particles are introduced into an electrochemical cell that includes luminol, (16). Upon the application of a potential step that reduces the quinone to hydroquinone, the electrocatalyzed reduction of O₂ to H₂O₂ proceeds. The resulting H₂O₂ mediates the HRP catalyzed oxidation of luminol with the concomitant emission of light.

The electrogenerated luminescence is observed only if the HRP labels bind to the telomerase units, and this occurs only provided telomerase (the analyte) exists in the analyzed cell extract. Also, the intensity of electrogenerated luminescence is controlled by the content of labels/avidin-HRP conjugates associated with the particles, and this is determined by the amount of telomerase enzyme in the sample. Furthermore, the rotation of the magnetic particles by means of the external rotating magnet further amplifies the emitted light intensity. Upon rotation of the particles, the electrocatalyzed reduction of O₂ and the interaction of H₂O₂ with luminol are controlled by convection rather than by diffusion, leading to enhanced (amplified) light emission.

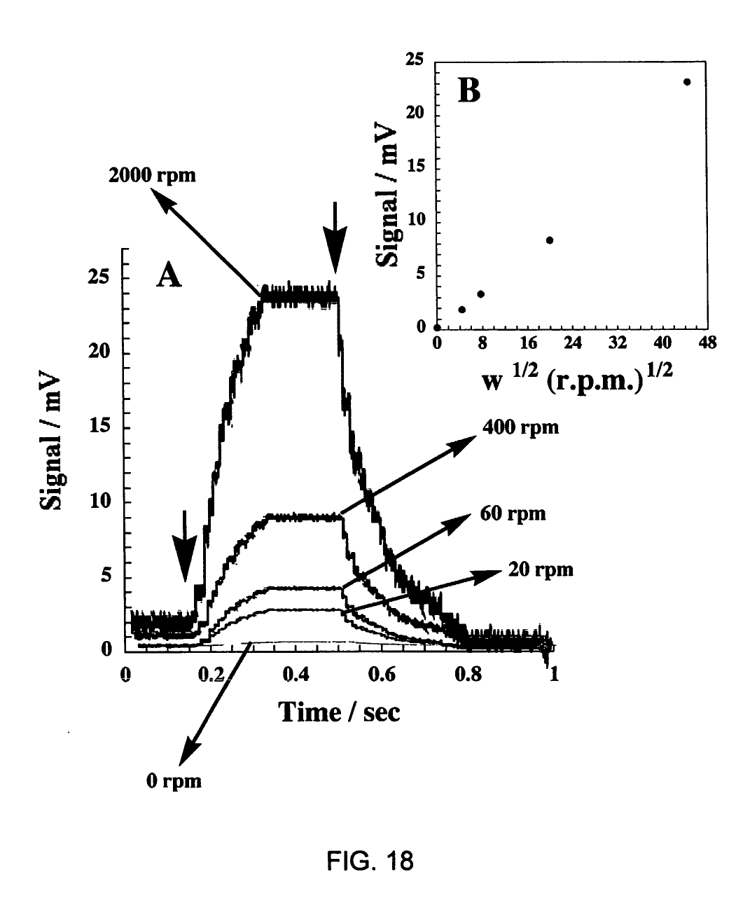

FIG. 18A depicts the analysis of 293-kidney cancer cells extract according to FIGS. 17A and B. It shows the light emitted from the system upon the analysis of a 100,000 cells extract dilution, while applying a potential step from 0 V to −0.5 V and rotating the particles at different speeds. The intensity of light emitted from the system is enhanced as the rotation speed increases. Provided that the functional magnetic particles behave in the analytical system as rotating microelectrodes, and that the electrocatalyzed generation of light is controlled by convection of the substrates to the rotating electrode, a linear dependency between the emitted light and ω^{1/2} (ω=rotation speed, rad·s⁻¹) should exist. FIG. 18B shows that indeed a linear relation between the electrogenerated light and ω^{1/2} exists. In control experiments where the naphthoquinone-functionalized magnetic particles or the avidin-HRP conjugate are excluded from the system, no light emission from the systems is detected at any rotation speed of the particles. These experiments confirm that the electrogenerated chemiluminescence originates from the primary electrocatalyzed reduction of O₂ to H₂O₂ and the subsequent HRP-mediated oxidation of luminol by H₂O₂.

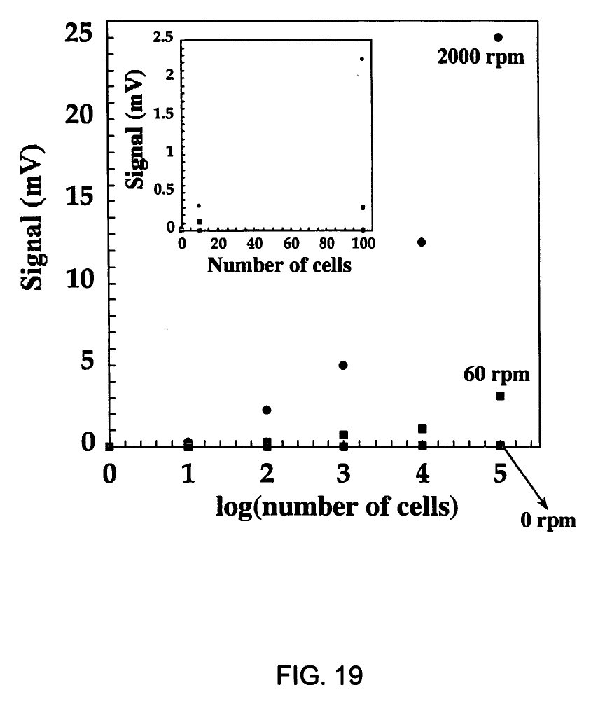

The electrogenerated chemiluminescence at constant rotation speed is controlled by the number of the cancer cells in the extract. FIG. 19 shows the light emitted from the electrochemical cell upon the analysis of different concentrations of the 293-kidney cancer cells at constant rotation speeds that corresponds to (i) 0 rpm, (ii) 60 rpm and (iii) 2000 rpm, and according to the detection route depicted in FIGS. 17A and B. In these systems, a potential step from 0.0 V to −0.5 V is applied on the functional particles. FIG. 19 inset, shows the light emitted from extracts that include 100 and 10 293-kidney cancer cells, respectively. For comparison, no generation of light is observed upon the application of the entire analysis protocol in the absence of cells or in the presence of 293-kidney cancer cells extract heated to 90° C. prior to the analysis process in order to inactivate the telomerase activity. This implies that no non-specific binding of the avidin-HRP conjugate to the magnetic particles or the electrode takes place.

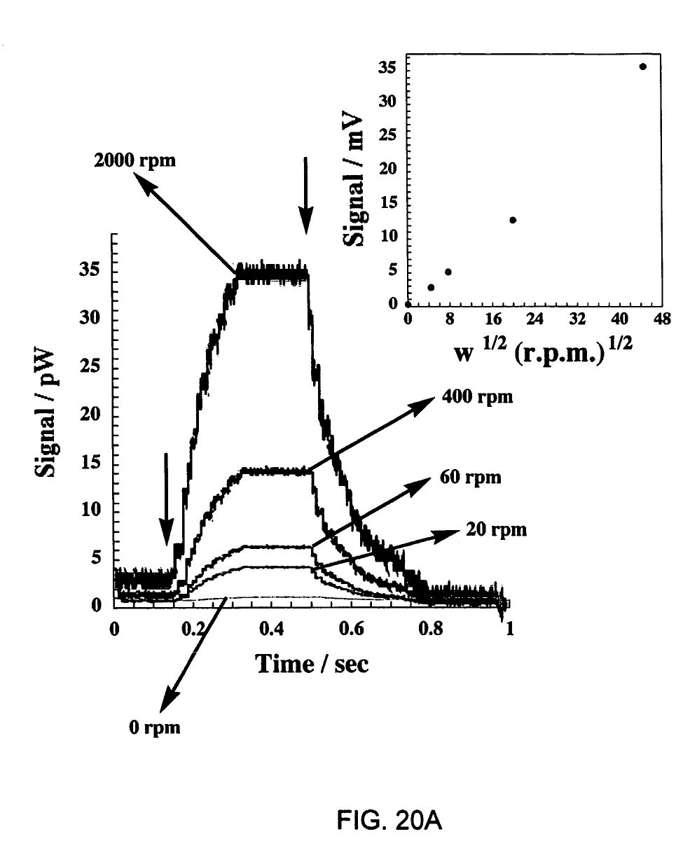

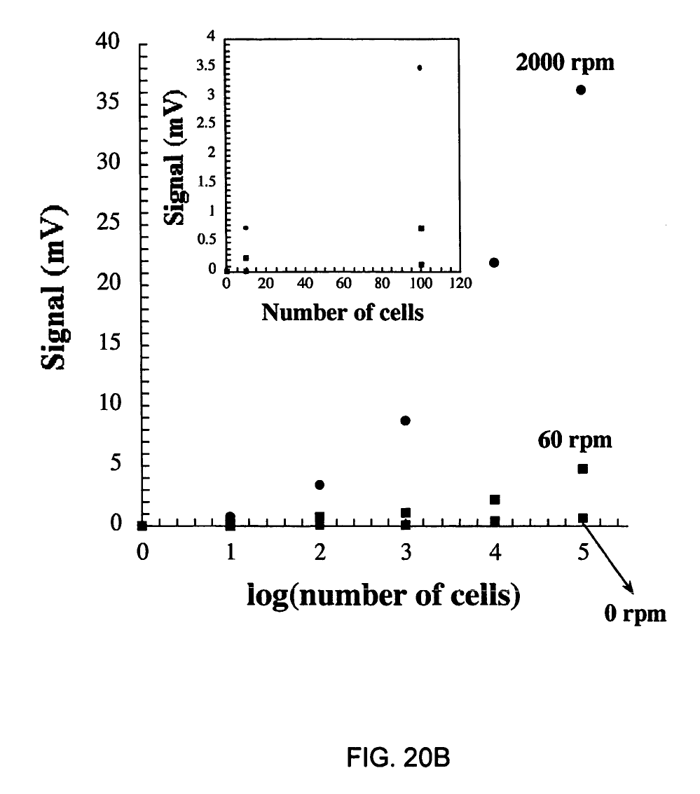

Similar results are observed upon the analysis of telomerase in cultured HeLa cells. FIG. 20A shows the electrogenerated chemiluminescence upon analysis of a HeLa cells extract, according to FIG. 17, and using different rotation speeds of the magnetic particles. The intensity of emitted light is enhanced upon increasing the rotation speed of the particles, and a linear relation between the intensity of generated light and ω^{1/2} (rad·s⁻¹) is observed, as showed in FIG. 20A, inset. The intensities of electrogenerated chemiluminescence upon the analysis of different concentrations of HeLa cells extracts are shown in FIG. 20B. In this experiment, the magnetic particles are rotated at a constant rotation speed of (i) 0 rpm, (ii) 60 rpm or (iii) 2000 rpm. Clearly, the light emitted upon the analysis of 10 HeLa cells is easily detectable.

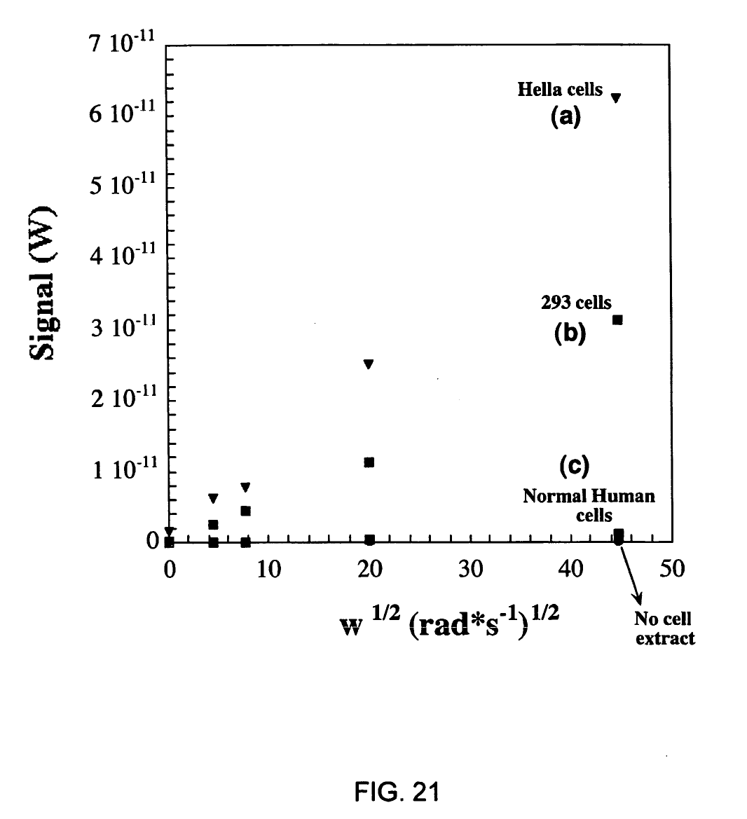

FIG. 21 shows the electrogenerated chemiluminescence upon analysis of telomerase activity HeLa cells extract, curve (a), a 293-kidney cancer cells extract, curve (b), and the analysis of a NHF (Normal Human fibroblast) cells extract, curve (c). Clearly, the electrogenerated chemiluminescence observed upon analyzing a 100-fold higher content of normal cells is ca. 200-fold lower than the light emitted from 1000 293 kidney cancer cells. The minute light emitted from the system that includes the normal NHF cells may be attributed to the non-specific adsorption of residual quantities of the avidin-HRP conjugate to the magnetic particles. The light generated in the system that includes the NHF cells may be considered as the background light level of the analysis scheme.

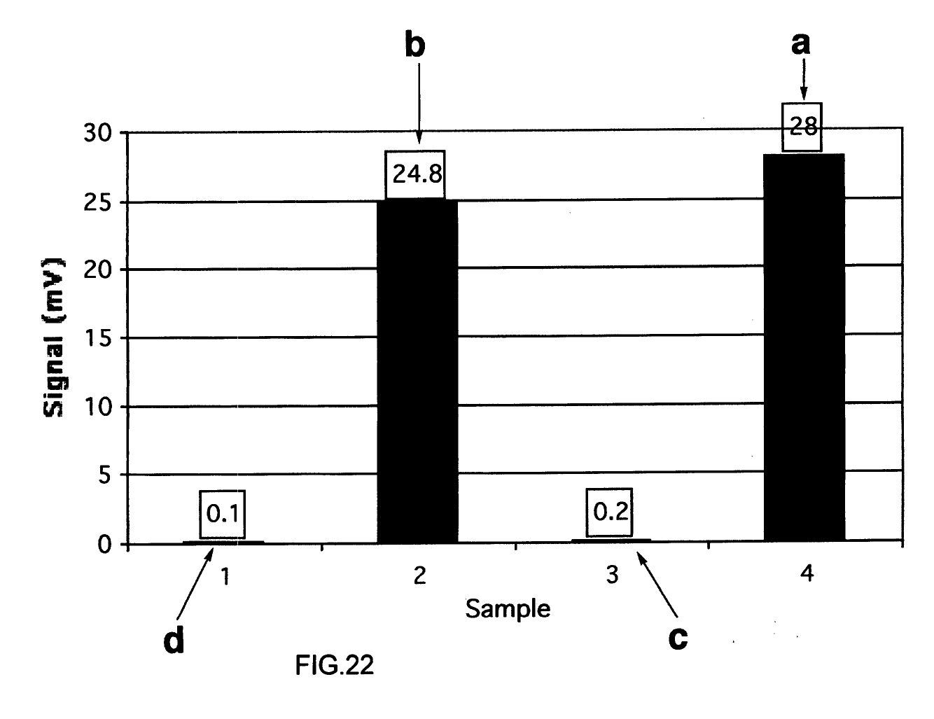

In addition, the capability to diagnose cancer in a suspected tissue is exemplified in FIG. 22. Different tissues from patients with lung cancer were assayed. The electrogenerated chemiluminescence intensities obtained upon analyzing healthy cells, adenocarcinoma and squamous epithelial carcinoma cells are shown in FIG. 22 and compared to the light signals observed upon analyzing healthy tissue or normal cell extracts. The chemiluminescence signals (telomerase activity) obtained from the carcinoma tissues were significantly higher than the minute chemiluminescence signal obtained from healthy tissue cells extracts.

This example clearly shows that the invention may be useful for the detection of telomerase as a rapid method to identify cancer cells and to monitor anti-cancer therapeutic treatments.

In all of the above telomerase assays, an Au-coated (50 nm gold layer) glass plate (Analytical-μSystem, Germany) was used as a working electrode (0.3 cm² area exposed to the solution). An auxiliary Pt electrode and a quasi-reference Ag electrode were made from wires of 0.5 mm diameter and added to the cell. The quasi-reference electrode was calibrated vs. saturated calomel electrode, and the potentials are given vs. SCE. An open electrochemical cell (230 μL) that includes the Au-electrode in a horizontal position and a light detection linked to a fiber optics, enabled easy light emission measurements upon application of the appropriate potential to the modified working electrode. The electrochemical measurements were performed using a potentiostat (EG&G, model 283) connected to a computer (EG&G Software 270/250 for). All the measurements were performed in 0.01 M phosphate buffer solution, pH 7.0, at room temperature. The electrochemically-induced chemiluminescence was measured with a light detection (Laserstat, Ophir) linked to an oscilloscope (Tektronix TDS 220). The light detector was connected to the electrochemical cell by an optical fiber. The background electrolyte solution was equilibrated with air and included luminol, 1×10⁻⁶ M.

1. A method for determining an analyte in an assayed sample, comprising:
(i) providing magnetic particles carrying a recognition agent that binds to or reacts with the analyte, such that, under assay conditions, said binding or reaction gives rise to a reaction that yields a reaction signal;
(ii) contacting said magnetic particles with the assayed sample, drawing the magnetic particles to a barrier surface through a magnet proximal to the barrier surface, providing the assay conditions and inducing the magnetic particles to rotate or vibrate in response to an external magnetic field that changes in time in a periodical manner, giving rise to a reaction signal that is enhanced during the rotation or vibration; and
(iii) reading said reaction signal.

2. The method according to claim 1, wherein said magnetic field is a rotating magnetic field.

3. The method according to claim 2, wherein said rotating magnetic field is induced by a rotating magnet.

4. The method according to claim 1, wherein said magnetic field is a vibrating magnetic field.

5. The method according to claim 1, wherein said reaction is a redox reaction.

6. The method according to claim 1, wherein said magnetic particles are confined to a support.

7. The method according to claim 1, wherein the recognition agent and the analyte react with one another in a manner to yield a reaction product.

8. The method according to claim 1, wherein the recognition agent is a catalyst that can induce a reaction in which the analyte is converted into a product.

9. The method according to claim 1, wherein the analyte and the recognition agent form a recognition pair and the detection of the analyte is based on the use of a reagent that binds to the formed pair.

10. The method according to claim 9, wherein the analyte is a protein analyte and the reagent is an antibody capable of binding to said analyte.

11. The method according to claim 9, wherein said analyte is a DNA analyte.

12. The method according to claim 11, wherein the assay conditions comprise a DNA polymerase and nucleotide bases, at least one of said nucleotide bases being bound to a detectable moiety.

13. The method according to claim 12, wherein said detectable moiety is biotin and the assay conditions further comprise an avidin bound enzyme.

14. The method according to claim 13, wherein the enzyme is horseradish peroxidase and the reaction signal is light emission.

15. The method according to claim 12, wherein the DNA polymerase is Taq Polymerase and the reaction conditions are such that enable polymerase chain reaction to take place.

16. The method according to claim 11, allowing the detection of at least one base mismatch.

17. The method according to claim 7, wherein the analyte is a catalyst that can induce a reaction in which the recognition agent is converted into a product.

18. The method according to claim 7, wherein the recognition agent comprises a catalyst that can induce a reaction in which the analyte is converted into a product.

19. The method according to claim 17, wherein the catalyst is an enzyme.

20. The method according to claim 19, wherein the enzyme is telomerase.

21. The method according to claim 20, wherein the assayed sample comprises cellular extract.

22. The method according to claim 1, wherein the analyte and the recognition agent form a recognition pair and the detection of the analyte is based on the use of a reagent that binds specifically to the analyte, where said analyte is first bound to the recognition agent.

23. The method according to claim 10, wherein said analyte is an antibody analyte.

24. The method according to claim 1, wherein at least one of the components of the chemical system remain during the analysis dissolved in the medium of the assayed sample.

25. The method according to claims 1, wherein the reaction signal is selected from electrical signal, light emission signal, calorimetric signal and formation of a precipitate.

26. A system for determining an analyte in an assayed sample, the system comprising:
(i) a cell with a barrier surface
(ii) a sub-system for causing the magnetic particles to rotate or vibrate, said subsystem comprising a motor associated with the magnet that causes the magnetic particles to rotate or vibrate;
(iii) magnetic particles having immobilized thereon a recognition agent such that in the presence of the analyte, a reaction occurs yielding a reaction signal, said signal being enhanced during the rotation or vibration of said magnet;
(iv) sensing member for sensing said reaction signal; and
(v) reader for reading said reaction signal.

27. The system according to claim 26, wherein said reaction is a redox reaction and said sensing member is an electrode.

28. The system according to claim 27, wherein said recognition agent comprises at least one molecule capable to transfer electrons between said electrode and said analyte.

29. The system according to claim 26, wherein the recognition agent and the analyte react with one another in a manner to yield a reaction product.

30. The system according to claim 26, wherein the analyte is a catalyst that can induce a reaction in which the recognition agent is converted into a product.

31. The system according to claim 26, wherein the recognition agent comprises a catalyst that can induce a reaction in which the analyte is converted into a product.

32. The system according to claim 26, wherein the analyte and the recognition agent form a recognition pair and the detection of the analyte is based on the use of a reagent that binds to the formed pair.

33. The system according to claim 26, wherein said analyte is a DNA analyte.

34. The system according to claim 26, wherein the analyte and the recognition agent form a recognition pair and the detection of the analyte is based on the use of a reagent that binds specifically to the analyte, where said analyte is first bound to the recognition agent.

35. The system according to claim 34, wherein said analyte is an antibody analyte.

36. The system according to claim 26, wherein said signal is selected from electrical signal, light emission signal, calorimetric signal and formation of a precipitate.

37. The method according to claim 20 for the detection of cancer cells.

38. The method according to claim 37 for the detection of cancer cells comprising:
(i) providing magnetic particles carrying a DNA recognition agent that serves as a primer for telomerase, such that, under assay conditions, the telomerase reaction enables a reaction that yields a reaction signal;
(ii) providing an assay sample comprising cellular extract from one or more cells suspected of being cancerous;
(iii) contacting said magnetic particles with the assayed sample, drawing the magnetic particles to a barrier surface through a magnet proximal to the barrier surface, providing the assay conditions and inducing the magnetic particles to rotate or vibrate, giving rise to a reaction signal that is enhanced during the rotation or vibration;
(iv) reading said reaction signal; and
(v) comparing said reading with a reading obtained from a control assay sample not containing cancerous cells, a higher reading in the assay sample than in the control assay sample indicating that said suspected cells are cancerous.

39. The method according to claim 38, wherein said reaction signal is light emission.

40. A method according to claim 1 for the detection of more than one analyte comprising:
(i) providing magnetic particles carrying more than one recognition agent, each of which recognition agents binds to or reacts with at least one of said analytes, such that, under assay conditions, each binding or reaction gives rise to a reaction that yields a distinguishable reaction signal, and in the presence of more than one of said analytes more than one distinguishable reaction signals are yielded;
(ii) contacting said magnetic particles with the assayed sample, drawing the magnetic particles to a barrier surface through a magnet proximal to the barrier surface, providing the assay conditions and inducing the magnetic particles to rotate or vibrate in response to an external magnetic field that changes in time in a periodical manner, giving rise to said distinguishable reaction signals; and
(iii) reading said distinguishable reaction signals.

41. The method of claim 40, wherein step (ii) comprises reading the distinguishable reaction signals using different reading means.

42. The method of claim 40, wherein steps (ii) and (iii) are repeated more than once, using different assay conditions.

43. A system for determining more than one analyte in an assayed sample, the system comprising:
(i) a cell with a barrier surface
(ii) a sub-system for causing the magnetic particles to rotate or vibrate;
(iii) magnetic particles having immobilized thereon more than one recognition agent such that in the presence of the analytes, reactions occur yielding distinguishable reaction signals, said signals being enhanced during the rotation or vibration of said magnet;
(iv) more than one sensing members for sensing each of said distinguishable reaction signals; and
(v) one or more readers for reading said reaction signal.

44. A system for determining an analyte in an assayed sample, the system comprising:
(i) a cell with a barrier surface
(ii) a sub-system for causing the magnetic particles to rotate or vibrate, said subsystem comprising a motor associated with the magnet that causes the magnetic particles to rotate or vibrate;
(iii) magnetic particles having immobilized thereon a recognition agent such that in the presence of the analyte, a reaction occurs yielding a reaction signal, said signal being enhanced during the rotation or vibration of said magnet; and
(iv) sensing member for sensing said reaction signal,
whereby the signal is indicative of the presence and/or amount of said analyte in the sample.

45. A system for determining more than one analyte in an assayed sample, the system comprising:
(i) a cell with a barrier surface
(ii) a sub-system for causing the magnetic particles to rotate or vibrate;
(iii) magnetic particles having immobilized thereon more than one recognition agent such that in the presence of the analytes, reactions occur yielding distinguishable reaction signals, said signals being enhanced during the rotation or vibration of said magnet; and
(iv) more than one sensing members for sensing each of said distinguishable reaction signals;
whereby each of said signals is indicative of the presence and/or amount of an analyte in the sample.
